(19) United States
(12) Patent Application Publication
(10) Pub. No.: US 2005/0144664 A1
(43) Pub. Date: Jun. 30, 2005

(54) Plant breeding method

(75) Inventors: Oscar S. Smith, Johnston, IA (US); Mark Cooper, Johnston, IA (US); Scott V. Tingey, Wilmington, DE (US); J. Antoni Rafalski, Wilmington, DE (US); Roy Luedtke, Ankeny, IA (US); William S. Niebur, Des Moines, IA (US)

Correspondence Address: QUINE INTELLECTUAL PROPERTY LAW GROUP, P.C., P O BOX 458, ALAMEDA, CA, 94501, US

(73) Assignee: Pioneer Hi-Bred International, INC., Johnston, IA (US)

(21) Appl. No.: 10/856,113

(22) Filed: May 27, 2004

Related U.S. Application Data
(60) Provisional application No. 60/474,359, filed on May 28, 2003.

(51) Int. Cl. A01H 1/00; C12N 15/82; A01H 5/00

(52) U.S. Cl. 800/266; 800/267; 800/298; 800/278

(57) ABSTRACT

Methods for using genetic marker genotype (e.g., gene sequence diversity information) to improve the process of developing plant varieties (e.g., single cross hybrids) with improved phenotypic performance are provided. Methods for predicting the value of a phenotypic trait in a plant are provided. The methods use genotypic, phenotypic, and optionally family relationship information for a first plant population to identify an association between at least one genetic marker and the phenotypic trait, and then use the association to predict the value of the phenotypic trait in one or more members of a second, target population of known marker genotype. Methods for identifying new allelic variants affecting the trait are also provided. Plants selected, provided, or produced by any of the methods herein, transgenic plants created by any of the methods herein, and digital systems for performing the methods herein are also provided.

### CROSS REFERENCE TO RELATED APPLICATIONS

This application is a non-provisional utility patent application claiming priority to and benefit of the following prior provisional patent application: U.S. Ser. No. 60/474,359, filed May 28, 2003, entitled “Plant Breeding Method” by Smith et al., which is incorporated herein by reference in its entirety for all purposes.

### FIELD OF THE INVENTION

The present invention provides a process for predicting the value of a phenotypic trait in a plant. The process uses genotypic, phenotypic, and family relationship information for a first plant population to identify an association between at least one genetic marker and the phenotypic trait, and then uses the association to predict the value of the phenotypic trait in members of a second, target population of known marker genotype. The invention also relates to a process for identifying new allelic variants affecting the phenotypic trait.

### BACKGROUND OF THE INVENTION

Selective breeding has been employed for centuries to improve, or attempt to improve, phenotypic traits of agronomic and economic interest in plants (e.g., yield, percentage of grain oil, and the like). In its most basic form, selective breeding involves selection of individuals as parents of the next generation on the basis of one or more phenotypic traits. However, such phenotypic selection is complicated by effects of the environment (e.g., soil type, rainfall, temperature range, and the like) on the expression of the phenotypic trait(s). Another problem with such phenotypic selection is that most phenotypic traits of interest are controlled by more than one genetic locus.

It has been estimated that 98% of the economically important phenotypic traits in domesticated plants are quantitative traits (U.S. Pat. No. 6,399,855 to Beavis, entitled “QTL mapping in plant breeding populations”). These traits are classified as oligogenic or polygenic based on the perceived numbers and magnitudes of segregating genetic factors affecting the variability in expression of the phenotypic trait.

Historically, the term quantitative trait has been used to describe variability in expression of a phenotypic trait that shows continuous variability and is the net result of multiple genetic loci possibly interacting with each other and/or with the environment. To describe a broader phenomenon, the term “complex trait” has been used to describe any trait that does not exhibit classic Mendelian inheritance attributable to a single genetic locus (Lander & Schork, Science 265: 2037 (1994)). The two terms are often used synonymously herein.

The development of ubiquitous polymorphic genetic markers (e.g., RFLPs, SNPS, or the like) that span the genome has made it possible for quantitative and molecular geneticists to investigate what Edwards, et al., in Genetics 115: 113 (1987) referred to as quantitative trait loci (QTL), as well as their numbers, magnitudes and distributions. QTL include genes that control, to some degree, qualitative and quantitative phenotypic traits that can be discrete or continuously distributed within a family of individuals as well as within a population of families of individuals.

Experimental paradigms have been developed to identify and analyze QTL (see, e.g., U.S. Pat. No. 5,385,835 to Helentjaris et al. entitled “Identification and localization and introgression into plants of desired multigenic traits,” U.S. Pat. No. 5,492,547 to Johnson entitled “Process for predicting the phenotypic trait of yield in maize,” and U.S. Pat. No. 5,981,832 to Johnson entitled “Process predicting the value of a phenotypic trait in a plant breeding program”). One such paradigm involves crossing two inbred lines to produce F1 single cross hybrid progeny, selfing the F1 hybrid progeny to produce segregating F2 progeny, genotyping multiple marker loci, and evaluating one to several quantitative phenotypic traits among the segregating progeny. The QTL are then identified on the basis of significant statistical associations between the genotypic values and the phenotypic variability among the segregating progeny. This experimental paradigm is ideal in that the parental lines of the F₁ generation have known linkage phases, all of the segregating loci in the progeny are informative, and linkage disequilibrium between the marker loci and the genetic loci affecting the phenotypic traits is maximized.

However, considerable resources must be devoted to determining the phenotypic performance of large numbers of hybrid and/or inbred progeny. Because the progeny from only two parents are studied, the experiments described above can only detect the trait loci (e.g., QTL) for which the two parents are polymorphic. This set of trait loci may only represent a fraction of the loci segregating in breeding populations of interest (e.g., breeding populations of maize, sorghum, soybean, canola, or the like, for example). In general, these progeny show variation for only one or a small number of the phenotypic traits that are of interest in applied breeding programs. This means that separate populations may need to be developed, scored for marker loci, and grown in replicated field experiments and scored for the phenotypic traits of interest. Additionally, methods used to detect QTL produce biased estimates of the QTL that are identified (see, e.g., Beavis (1994) “The power and deceit of QTL experiments: Lessons from comparative QTL studies” in Wilkinson (ed.) Proc. 49^{th} Ann. Corn and Sorghum Res. Conf., American Seed Trade Assoc, Chicago, Ill., pp 250-266). Additional imprecision is introduced in extrapolating the identification of QTL to the progeny of genetically different parents within a breeding population. Furthermore, many if not all traits are affected by environmental factors, which can also introduce imprecision.

The present invention overcomes the above noted difficulties, for example, by identifying QTL-associated genetic markers through an association analysis that can accommodate complex plant populations (in which larger numbers of genetic loci affecting the phenotype for multiple traits of interest are expected to be segregating, as compared to bi-parental populations), take advantage of information generated by existing breeding programs, and optionally account for environmental effects, and by applying this information to predict phenotypes, e.g., of hybrid progeny. A complete understanding of the invention will be obtained upon review of the following.

### SUMMARY OF THE INVENTION

The present invention provides a process for predicting the value of a phenotypic trait in a plant. The process uses genotypic, phenotypic, and family relationship information for a first plant population to identify an association between at least one genetic marker and the phenotypic trait, and then uses the association to predict the value of the phenotypic trait in members of a second, target population of known marker genotype. The invention also relates to a process for identifying new allelic variants affecting the phenotypic trait.

Thus, a first general class of embodiments provides methods of predicting a value of a phenotypic trait in a target plant population. In the methods, an association between at least one genetic marker and the phenotypic trait is provided. For example, an association between the phenotypic trait and a haplotype comprising two or more genetic markers can be provided. The association is evaluated in a first plant population which is an established breeding population or a portion thereof. The association is evaluated in the first plant population according to a statistical model that incorporates a genotype of the first plant population for a set of genetic markers and a value of the phenotypic trait in the first plant population. The statistical model can also incorporate family relationships among the members of the first plant population. The value of the phenotypic trait in at least one member of the target plant population is then provided. The value is predicted from the association and from a genotype of the at least one member for the at least one genetic marker associated with the phenotypic trait, e.g., by using both pedigree and genetic marker information.

In one class of embodiments, the first plant population comprises a plurality of inbreds, single cross F1 hybrids, or a combination thereof. For example, the first plant population optionally consists of inbreds, single cross F1 hybrids, or a combination thereof. Since the members of the first plant population are members of an established breeding population, the ancestry of each inbred and/or single cross F1 hybrid is typically known, and each inbred and/or single cross F1 hybrid is typically a descendent of at least one of three or more founders. Since the members of the first plant population typically come from an established breeding population with a multi-generation pedigree, the members of the first plant population optionally span multiple breeding cycles (e.g., at least three, at least four, at least five, at least seven, or at least nine breeding cycles). The established breeding population itself typically comprises at least three founders (e.g., at least 10 founders, at least 50 founders, at least 100 founders, or at least 200 founders, e.g., between about 100 and about 200 founders) and descendents of the founders, wherein the ancestry of the descendents is known. The first plant population can comprise essentially any number of members, e.g., between about 50 and about 5000.

The phenotypic trait can be, e.g., a qualitative trait, a quantitative trait, a single gene trait, a multigenic trait, and/or the like. The value of the phenotypic trait in the first plant population is obtained, e.g., by evaluating the phenotypic trait among the members of the first plant population. The phenotype can be evaluated in the members of first plant population (e.g., the inbreds and/or single cross F1 hybrids comprising the first plant population). Alternatively, the value of the phenotypic trait in the first plant population can be obtained by evaluating the phenotypic trait among the members of the first plant population in at least one topcross combination with at least one tester parent. Phenotypic traits include, but are not limited to, yield, grain moisture content, grain oil content, root lodging resistance, stalk lodging resistance, plant height, ear height, disease resistance, insect resistance, drought resistance, grain protein content, test weight, and cob color.

The set of genetic markers can comprise essentially any convenient number and type of genetic markers. For example, the set of genetic markers can comprise one or more of: a single nucleotide polymorphism (SNP), a multinucleotide polymorphism, an insertion or a deletion of at least one nucleotide (indel), a simple sequence repeat (SSR), a restriction fragment length polymorphism (RFLP), a random amplified polymorphic DNA (RAPD) marker, or an arbitrary fragment length polymorphism (AFLP). The set of genetic markers can comprise, for example, between 1 and 50,000 (or even more) genetic markers; e.g., between one and ten markers or between 500 and 50,000 markers. The genotype of the first plant population for the set of genetic markers can be experimentally determined and/or predicted. Similarly, the genotype of the members of the target plant population for the set of genetic markers can be experimentally determined and/or predicted.

In a preferred class of embodiments, the association between the at least one genetic marker and the phenotypic trait is evaluated by performing Bayesian analysis using a linear model, a mixed linear model, or a nonlinear model. In one such preferred class of embodiments, the association is evaluated by performing Bayesian analysis using a linear model, the Bayesian analysis being implemented via a reversible jump Markov chain Monte Carlo algorithm. Typically, the Bayesian analysis is implemented via a computer program or system. In another preferred class of embodiments, the association is evaluated by performing a transmission disequilibrium test.

The target plant population can comprise inbred plants, hybrid plants, or a combination thereof. In a preferred class of embodiments, the target plant population comprises hybrid plants that comprise F1 progeny produced from single crosses between inbred lines. These F1 progeny can be produced, e.g., from single crosses between inbred progeny comprising the first plant population and/or new inbreds. Similarly, the target plant population can comprise an advanced generation produced from breeding crosses involving at least one of the members of the first plant population.

The value of the phenotypic trait in the at least one member of the target plant population can be predicted by any of a variety of methods. For example, for simple qualitative traits, the phenotype can be predicted from the identity of the genetic marker allele(s) found in the member(s) of the target plant population. As other examples, the value of the phenotypic trait in the at least one member of the target plant population can be predicted using a best linear unbiased prediction method, a multiple regression method, a selection index technique, a ridge regression method, a linear optimization method, or a non-linear optimization method.

The first and target plant populations can comprise essentially any type of plants. For example, in a preferred class of embodiments, the first and target plant populations comprise (e.g., consist of) diploid plants, including, but not limited to, hybrid crop plants, such as maize (e.g., Zea mays), soybean, sorghum, wheat, sunflower, rice, canola, cotton, and millet, for example.

The methods optionally include selecting at least one of the members of the target plant population having a desired predicted value of the phenotypic trait. The at least one selected member of the target plant population can be bred with at least one other plant or selfed, e.g., to create a new line or hybrid having a desired value of the phenotypic trait. In another class of embodiments, the methods include cloning a gene that is linked to the at least one genetic marker associated with the phenotypic trait, wherein expression of the gene affects the phenotypic trait, and optionally include constructing a transgenic plant by expressing the cloned gene in a host plant.

Another general class of embodiments provides methods of selecting a plant. In the methods, an association between at least one genetic marker and the phenotypic trait is provided. The association is evaluated in a first plant population which is an established breeding population or a portion thereof. The association is evaluated in the first plant population according to a statistical model that incorporates a genotype of the first plant population for a set of genetic markers and a value of the phenotypic trait in the first plant population. The statistical model can also incorporate family relationships among the members of the first plant population. One or more plants from one or more non-adapted lines are then provided. The one or more plants are selected for a selected genotype comprising the at least one genetic marker associated with the phenotypic trait. The selected genotype optionally comprises at least one allele of at least one of the genetic markers associated with the phenotypic trait that is novel with respect to the genetic marker alleles found in the first population.

A novel genetic marker genotype can indicate the presence of a novel allele of a QTL associated with the genetic marker (and with the phenotypic trait). To determine if this putative novel QTL allele is one that favorably affects the phenotypic trait, the methods can include evaluating the phenotypic trait in the one or more plants having the selected genotype. At least one plant having the selected genotype and a desirable value of the phenotypic trait can be selected. In addition, the at least one selected plant having the selected genotype and the desirable value of the phenotypic trait can be bred with at least one other plant (e.g., to introduce the genetic marker allele and thus the putative novel QTL allele into the adapted germplasm).

In a preferred class of embodiments, the association between the at least one genetic marker and the phenotypic trait is evaluated by performing Bayesian analysis using a linear model, a mixed linear model, or a nonlinear model. In one such preferred class of embodiments, the association is evaluated by performing Bayesian analysis using a linear model, the Bayesian analysis being implemented via a reversible jump Markov chain Monte Carlo algorithm. In another preferred class of embodiments, the association is evaluated by performing a transmission disequilibrium test.

All of the various optional configurations and features noted for the embodiments above apply here as well, to the extent they are relevant, e.g., for composition of the first plant population and/or the established breeding population, types of phenotypic traits, types and number of genetic markers, and the like.

Plants selected, provided, or produced by any of the methods herein form another feature of the invention, as do transgenic plants created by any of the methods herein. Digital systems for practicing the methods or aspects thereof are also provided. Kits comprising system components, plants selected by the methods, or both, along with appropriate containers, packaging materials, instructions for practicing the methods, or the like, are also a feature of the invention.

### BRIEF DESCRIPTION OF THE DRAWINGS

FIG. 1 is a pedigree schematically illustrating the relationships between various inbred lines and single cross hybrids in an example of a portion of an established breeding population (or an example first plant population).

FIG. 2 provides a schematic overview of a typical pedigree corn breeding program.

FIG. 3 schematically illustrates a software implementation of a Bayesian analysis.

FIG. 4 depicts a plot of the TDT likelihood ratio statistic for cob color for 511 markers ordered by their position on chromosome 1.

### Definitions

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a protein” includes two or more proteins; reference to “a cell” includes mixtures of cells, and the like.

An “allele” or “allelic variant” is any of one or more alternative forms of a gene or genetic marker. In a diploid cell or organism, the two alleles of a given gene (or marker) typically occupy corresponding loci on a pair of homologous chromosomes.

The term “association” or “associated with” in the context of this invention refers to one or more genetic marker alleles and phenotypic trait alleles that are in linkage disequilibrium, i.e., the marker genotypes and trait phenotypes are found together in the progeny of a plant or plants more often than if the marker genotypes and trait phenotypes segregated independently.

A “breeding cycle” describes the separation between two inbred parents and an inbred offspring of these parents. A breeding cycle can include, for example, crossing two inbred lines to produce an F1 hybrid, selfing the F1 hybrid, and selfing several more times to produce the inbred offspring. A breeding cycle optionally includes one or more backcrosses to one of the inbred parents. The separation between an inbred and a single cross F1 hybrid or between two single cross F1 hybrids can also be described in terms of breeding cycles. To determine the breeding cycle distance of a single cross F1 hybrid to an inbred, the breeding cycle difference between the inbred and each inbred parent of the hybrid is determined; the larger of these two numbers is the number of breeding cycles separating the F1 single cross hybrid and the inbred. To determine the breeding cycle distance of a first single cross F1 hybrid to a second single cross F1 hybrid, all possible combinations of the first hybrid's inbred parents with the second hybrid's inbred parents are compared to each other, and the breeding cycle distance between the two hybrids equals the largest distance between any one of these combinations of inbred parents.

A “diploid plant” is a plant that has two sets of chromosomes, typically one from each of its two parents.

An “established breeding population” is a collection of plants produced by and/or used as parents in a breeding program, e.g., a commercial breeding program. The members of the established breeding population have typically been well-characterized; for example, several phenotypic traits of interest may have been evaluated, e.g., under different environmental conditions, at multiple locations, and/or at different times.

“F₁” refers to the first filial generation, the progeny of a mating between two individuals or between two inbred lines. “Advanced generations” are the F₂, F₃, and later generations produced from the F₁ progeny by selfing or sexual crosses (e.g., with other F₁ progeny, with an inbred line, etc.).

A “founder” is an inbred or single cross F1 hybrid that contains one or more alleles (e.g., genetic marker alleles) that can be tracked through the founder's descendents in a pedigree of a population, e.g., a breeding population. In an established breeding population, for example, the founders are typically (but not necessarily) the earliest developed lines.

The term “gene” is used broadly to refer to any nucleic acid associated with a biological function. Genes typically include coding sequences and/or regulatory sequences required for expression of such coding sequences.

A “genetic marker” is a nucleotide or a polynucleotide sequence that is present in a plant genome and that is polymorphic in a population of interest, or the locus occupied by the polymorphism, depending on context. Genetic markers include, for example, SNPs, indels, SSRs, RFLPs, RAPDs, and AFLPs, among many other examples. Genetic markers can, e.g., be used to locate on a chromosome genetic loci containing alleles which contribute to variability in expression of phenotypic traits. Genetic markers also refer to polynucleotide sequences complementary to the genomic sequences, such as sequences of nucleic acids used as probes.

“Genotype” refers to the genetic constitution of a cell or organism. An individual's “genotype for a set of genetic markers” consists of the specific alleles, for one or more genetic marker loci, present in the individual.

“Germplasm” is the totality of the genotypes of a population or other group of individuals (e.g., a species). Germplasm can also refer to plant material, e.g., a group of plants that act as a repository for various alleles. “Adapted germplasm” refers to plant materials of proven genetic superiority, e.g., for a given environment or geographical area, while “non-adapted germplasm,” “raw germplasm,” or “exotic germplasm” refers to plant materials of unknown or unproven genetic value, e.g., for a given environment or geographical area; as such, non-adapted germplasm refers to plant materials that are not part of an established breeding population and that do not have a known relationship to a member of the established breeding population.

A “haplotype” is the set of alleles an individual inherited from one parent. A diploid individual thus has two haplotypes. The term haplotype is often used in a more limited sense to refer to physically linked and/or unlinked genetic markers (e.g., sequence polymorphisms) associated with a phenotypic trait. A “haplotype block” (sometimes also referred to in the literature simply as a haplotype) is a group of two or more genetic markers that are physically linked on a single chromosome (or a portion thereof). Typically, each block has a few common haplotypes, and a subset of the genetic markers (i.e., a “haplotype tag”) can be chosen that uniquely identifies each of these haplotypes.

The phrase “high throughput screening” refers to assays in which the format allows large numbers of genetic markers (e.g., nucleic acid sequences), large numbers of individual or pools of genotypes, or both, to be screened. In the context of the instant invention, high throughput screening is the screening of large numbers of genotypes as individuals or pools for nucleic acid sequences of the plant genome to identify the presence of genetic marker alleles.

A “hybrid,” “hybrid plant,” or “hybrid progeny” is an individual produced from genetically different parents (e.g., a genetically heterozygous or mostly heterozygous individual). Typically, the parents of a hybrid differ in several important respects. Hybrids are often more vigorous than either parent, but they cannot breed true.

If two individuals possess the same allele at a particular locus, the alleles are “identical by descent” if the alleles were inherited from one common ancestor (i.e., the alleles are copies of the same parental allele). The alternative is that the alleles are “identical by state” (i.e., the alleles appear the same but are derived from two different copies of the allele). Identity by descent information is useful for linkage studies; both identity by descent and identity by state information can be used in association studies such as those described herein, although identity by descent information can be particularly useful.

An “inbred line” of plants is a genetically homozygous or nearly homozygous population. An inbred line, for example, can be derived through several cycles of selfing. Inbred lines breed true, e.g., for one or more phenotypic traits of interest. An “inbred,” “inbred plant,” or “inbred progeny” is a plant sampled from an inbred line.

“Linkage” refers to the tendency of alleles at different loci on the same chromosome to segregate together more often than expected by chance if their transmission were independent, as a consequence of their physical proximity.

The phrase “linkage disequilibrium” (also called “allelic association”) refers to a phenomenon wherein particular alleles at two or more loci tend to remain together in linkage groups when segregating from parents to offspring with a greater frequency than expected from their individual frequencies in a given population. For example, a genetic marker allele and a QTL allele show linkage disequilibrium when they occur together with frequencies greater than those predicted from the individual allele frequencies. It is worth noting that linkage refers to a relationship between loci, while linkage disequilibrium refers to a relationship between alleles.

A “locus” is a position on a chromosome (e.g., of a gene, a genetic marker, or the like).

The term “nucleic acid” encompasses any physical string of monomer units that can be corresponded to a string of nucleotides, including a polymer of nucleotides (e.g., a typical DNA or RNA polymer), PNAs, modified oligonucleotides (e.g., oligonucleotides comprising bases that are not typical to biological RNA or DNA, such as 2′-O-methylated oligonucleotides), and the like. A nucleic acid can be e.g., single-stranded or double-stranded. Unless otherwise indicated, a particular nucleic acid sequence of this invention optionally comprises or encodes complementary sequences, in addition to any sequence explicitly indicated.

A “pedigree” is a record of the ancestor lines, individuals, or germplasm for an individual or a family of related individuals.

The phrase “phenotypic trait” refers to the appearance or other detectable characteristic of a plant, resulting from the interaction of its genome with the environment.

The term “plurality” refers to more than half of the whole. For example, a plurality of a population is more than half the members of that population.

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

A “plant population” is a collection of plants. The collection includes at least two plants, and can include, for example, 10 or more, 50 or more, 100 or more, 500 or more, 1000 or more, or even 5000 or more plants. The members of the population can be related and/or unrelated to each other; for example, the plants can have known pedigree relationships to each other.

The term “progeny” refers to the descendant(s) of a particular plant (selfcross) or pair of plants (cross-pollinated). The descendant(s) can be, for example, of the F1, the F₂, or any subsequent generation.

A “qualitative trait” is a phenotypic trait that is controlled by one or a few genes that exhibit major phenotypic effects. Because of this, qualitative traits are typically simply inherited. Examples include, but are not limited to, flower color, cob color, and disease resistance such as Northern corn leaf blight resistance.

A “quantitative trait” is a phenotypic trait that can be described numerically (i.e., quantitated or quantified). A quantitative trait typically exhibits continuous variation between individuals of a population; that is, differences in the numerical value of the phenotypic trait are slight and grade into each other. Frequently, the frequency distribution in a plant population of a quantitative phenotypic trait exhibits a bell-shaped curve. A quantitative trait is typically the result of a genetic locus interacting with the environment or of multiple genetic loci (QTL) interacting with each other and/or with the environment. Examples of quantitative traits include plant height and yield.

The term “quantitative trait locus” (“QTL”) or the term “marker trait association” refers to an association between a genetic marker and a chromosomal region and/or gene that affects the phenotype of a trait of interest. Typically, this is determined statistically, e.g., based on one or more methods published in the literature. A QTL can be a chromosomal region and/or a genetic locus with at least two alleles that differentially affect the expression of a phenotypic trait (either a quantitative trait or a qualitative trait).

The phrase “sexually crossed” or “sexual reproduction” in the context of this invention refers to the fusion of gametes to produce seed by pollination. A “sexual cross” or “cross-pollination” is pollination of one plant by another. “Selfing” is the production of seed by self-pollinization, i.e., pollen and ovule are from the same plant.

A “single cross F1 hybrid” is an F₁ hybrid produced from a cross between two inbred lines.

A “tester” is a line or individual plant with a standard genotype, known characteristics, and established performance. A “tester parent” is a plant from a tester line that is used as a parent in a sexual cross. Typically, the tester parent is unrelated to and genetically different from the plant(s) to which it is crossed. A tester is typically used to generate F1 progeny when crossed to individuals or inbred lines for phenotypic evaluation.

The phrase “topcross combination” refers to the process of crossing a single tester line to multiple lines. The purpose of producing such crosses is to determine phenotypic performance of hybrid progeny; that is, to evaluate the ability of each of the multiple lines to produce desirable phenotypes in hybrid progeny derived from the line by the tester cross.

A “transgenic plant” is a plant into which one or more exogenous polynucleotides have been introduced by any means other than sexual cross or selfing. Examples of means by which this can be accomplished are described below, and include Agrobacterium-mediated transformation, biolistic methods, electroporation, in planta techniques, and the like. Transgenic plants may also arise from sexual cross or by selfing of transgenic plants into which exogenous polynucleotides have been introduced.

A “variety” is a subdivision of a species for taxonomic classification. “Variety” is used interchangeably with the term “cultivar” to denote a group of individuals that are genetically distinct from other groups of individuals in a species. An agricultural variety is a group of similar plants that can be identified from other varieties within the same species by structural features and/or performance.

A variety of additional terms are defined or otherwise characterized herein.

### DETAILED DESCRIPTION

Association studies provide an alternative approach to identifying chromosomal regions and/or genes affecting phenotypes of interest using genetic linkage. In brief, while linkage studies attempt to identify QTL that co-segregate with a phenotypic trait within one or more families, association studies typically attempt to identify QTL by identifying particular allelic variants that are associated with the phenotypic trait in a population (not necessarily a bi-parental family). An allelic variant identified as being associated with the trait can be, e.g., an allelic variant of a genetic marker that is in linkage disequilibrium with a functional variant (an allele of a gene that affects the phenotypic trait), or the genetic marker and the functional variant can be synonymous (e.g., a SNP in a coding region that results in an altered activity of the encoded protein).

Linkage disequilibrium is a phenomenon observed in populations in which particular alleles at two (or more) loci occur together at a frequency greater than the product of the two (or more) allele frequencies. For example, assume that a mutation at locus A occurs to produce new allele Aₘ on a chromosome bearing allele Bₙ at locus B. If no recombination occurs between loci A and B, the haplotype AₘBₙ is preserved. If recombination between the loci occurs, the haplotype is not preserved. Eventually, as recombination occurs through multiple generations, the new allele Aₘ would occur with the other alleles of B in proportion to their relative frequency (that is, eventually linkage equilibrium is achieved). In the first segregating generation of a cross of two populations or genotypes, however, the frequency of haplotype AₘBₙ is greater than the product of the Aₘ allele frequency and the Bₙ allele frequency; i.e., linkage disequilibrium is observed. The approach to equilibrium is a function of the recombination frequency in a randomly mating population. For unlinked loci, the haplotype frequency goes halfway to the equilibrium value each generation; the more tightly the loci are linked, the longer the disequilibrium persists in the population. Association studies taking advantage of linkage disequilibrium can thus incorporate many past generations of recombination to achieve high-resolution, fine scale gene localization (see, e.g., Xiong and Guo (1997) “Fine-scale mapping of quantitative trait loci using historical recombinations” Genetics 145: 1201-1218).

Design and execution of various types of association studies have been described in the art; see, e.g., Rao and Province, eds., (2001) Advances in Genetics volume 42, Genetic Dissection of Complex Traits; Balding et al., eds. (2001) Handbook of Statistical Genetics, John Wiley and Sons Ltd.; Borecki and Suarez (2001) “Linkage and association: basic concepts” Adv Genet 42: 45-66; Cardon and Bell (2001) “Association study designs for complex diseases” Nat Rev Genet 2: 91-99; and Risch (2000) “Searching for genetic determinants for the new millennium” Nature 405: 847-856. Association studies have been used both to evaluate candidate genes for association with a phenotypic trait (e.g., Thornsberry et al. (2001) “Dwarf8 polymorphisms associate with variation in flowering time” Nature Genetics 28: 286-289) and to perform whole genome scans to identify genes that contribute to phenotypic variation (e.g., Paunio et al. (2001) “Genome-wide scan in a nationwide study sample of schizophrenia families in Finland reveals susceptibility loci on chromosomes 2q and 5q” Human Molecular Genetics 10: 3037-3048 and Liu et al. (2002) “Genomewide linkage analysis of celiac disease in Finnish families” Am. J. Hum. Genet. 70: 51-59).

As will be evident, linkage disequilibrium must exist in the region(s) of interest for association studies to be powerful (if no linkage disequilibrium exists, an association study can identify only a marker that is itself an actual functional variant). The rate at which (number of base pairs over which) linkage disequilibrium declines thus affects the resolution of an association study and the number of markers required. Such considerations can, for example, affect the choice of population to be used in the analysis. A number of studies have examined linkage disequilibrium in humans (e.g., Reich et al. (2001) “Linkage disequilibrium in the human genome” Nature 411: 199-204 and Daly et al. (2001) “High-resolution haplotype structure in the human genome” Nature Genetics 29: 229-232). Linkage disequilibrium has also been analyzed in plants; for example, a recent study by the authors and others indicates that strong linkage disequilibrium between SNP loci extends at least 500 bp in maize (Ching et al. (2002) “SNP frequency, haplotype structure and linkage disequilibrium in elite maize inbred lines” BMC Genetics 3: 19; see also Remington et al. (2001) “Structure of linkage disequilibrium and phenotypic associations in the maize genome” Proc. Natl. Assoc. Sci. 98: 11479-11484; Tenaillon et al. (2001) “Patterns of DNA sequence polymorphism along chromosome 1 of maize” Proc Natl Acad Sci USA 98: 9161-9166; and Jannoo et al. (1999) “Linkage disequilibrium among modern sugarcane cultivars” Theor App Genet 99: 1053-1060).

Although a number of association studies involving humans and animals have been performed (see, e.g., Paunio et al. (2001) “Genome-wide scan in a nationwide study sample of schizophrenia families in Finland reveals susceptibility loci on chromosomes 2q and 5q” Human Molecular Genetics 10: 3037-3048; Liu et al. (2002) “Genomewide linkage analysis of celiac disease in Finnish families” Am. J. Hum. Genet. 70: 51-59; Terwilliger (2001) “On the resolution and feasibility of genome scanning approaches” Adv. Genet. 42: 351-391; and Grupe et al. (2001) “In silico mapping of complex disease-related traits in mice” Science 292: 1915-1918), fewer studies have been performed involving plants. Plant pedigrees present several challenges that require modification or extension of methods used for humans and animals (see, e.g., Yi and Xu (2001) “Bayesian mapping of quantitative trait loci under complicated mating designs” Genetics 157: 1759-1771). For example, QTL mapping methods applicable to plants may need to deal with both selfing and sexual crossing, pure inbred lines as breeding population founders, and large family sizes.

Bayesian methods have been proposed for association studies in plants that account for these factors. For example, Yi and Xu (2001) “Bayesian mapping of quantitative trait loci under complicated mating designs” Genetics 157: 1759-1771 and Bink et al. (2002) “Multiple QTL mapping in related plant populations via a pedigree-analysis approach” Theor. Appl. Genet. 104: 751-762 describe Bayesian methods for QTL mapping in complex plant populations. These methods incorporate genotypic, phenotypic, and family pedigree information for complex plant populations (e.g., a first plant population). Use of such complex populations offers a number of advantages. For example, a large number of single cross hybrids (or a large number of segregating F2 progeny from a biparental cross, or the like) need not be generated and phenotyped to perform the analysis; instead, plants and/or lines can be chosen from the breeding population, where phenotypic evaluation of large numbers of progeny of different types is a normal part of the breeding program. Breeding programs typically evaluate the phenotypes of a large number of progeny, often replicated at two or more locations (thus providing data on environmental effects). Since considerable time and effort is required to accurately assess most of the economically important phenotypic traits, using data generated as part of an ongoing breeding program offers considerable time and cost savings as well as potentially more reliable phenotypic data and thus a better map. See, e.g., Rafalski (2002) “Applications of single nucleotide polymorphisms in crop genetics” Curr. Opin. Plant Bio. 5: 94-100 and Rafalski (2002) “Novel genetic mapping tools in plants: SNPs and LD-based approaches” Plant Sci 162: 329-333.

The present invention provides methods for using genetic marker genotype, phenotypic information, and family relationship data for plants in a first plant population (e.g., a breeding population or a subset thereof) to identify an association between at least one genetic marker and a phenotypic trait, for example, using Bayesian methods such as those referenced above. The methods include prediction of the value of the phenotypic trait in one or more members of a second, target plant population based on their genotype for the one or more genetic markers associated with the trait.

The methods have a number of applications, e.g., in applied breeding programs in plants (e.g., hybrid crop plants; similar methods can be applied for animals). For example, the methods can be used to predict the phenotypic performance of hybrid progeny, e.g., a single cross hybrid produced (actually or hypothetically) by crossing a given pair of inbred lines of known marker genotype. Similarly, by allowing prediction of phenotypic performance of the potential progeny from a cross, the methods can facilitate selection of plants (e.g., inbred plants, hybrid plants, etc.) for use as parents in one or more crosses; the methods permit selection of parental plants whose offspring have the highest probability of possessing the desired phenotype.

A first general class of embodiments provides methods of predicting a value of a phenotypic trait in a target plant population. In the methods, an association between at least one genetic marker and the phenotypic trait is provided. The association is evaluated in a first plant population, which first plant population is an established breeding population or a portion thereof. The association is evaluated in the first plant population according to a statistical model that incorporates a genotype of the first plant population for a set of genetic markers and a value of the phenotypic trait in the first plant population. The value of the phenotypic trait in at least one member of the target plant population is then provided. The value is predicted from the association and from a genotype of the at least one member for the at least one genetic marker associated with the phenotypic trait. The value is typically predicted in advance of or instead of experimentally determining the value.

The phenotypic trait can be a quantitative trait, e.g., for which a quantitative value is provided. Alternatively, the phenotypic trait can be a qualitative trait, e.g., for which a qualitative value is provided. The trait can be determined by a single gene, or it can be determined by two or more genes.

The methods optionally include selecting at least one of the members of the target plant population having a desired predicted value of the phenotypic trait, and optionally also include breeding at least one selected member of the target plant population with at least one other plant (or selfing the at least one selected member, e.g., to create an inbred line).

The first plant population typically comprises a plurality of inbreds, single cross F1 hybrids, or a combination thereof. For example, in one class of embodiments, the first plant population comprises a plurality of inbreds. In another class of embodiments, the first plant population comprises a plurality of single cross F1 hybrids. In yet another class of embodiments, the first plant population comprises a plurality of a combination of inbreds and single cross F1 hybrids. The first plant population optionally consists of inbreds, single cross F1 hybrids, or a combination thereof. The inbreds can be from inbred lines that are related and/or unrelated to each other, and the single cross F1 hybrids can be produced from single crosses of said inbred lines and/or one or more additional inbred lines.

As noted, the members of the first plant population are sampled from an existing, established breeding population (e.g., a commercial breeding population). The members of an established breeding population are typically descendents of a relatively small number of founders and are thus typically highly inter-related. The ancestry of each member other than the founders is generally known. Thus, for example, an established breeding population can comprise at least three founders and their descendents, where the ancestry of the descendents is known (e.g., at least 10 founders, at least 50 founders, at least 100 founders, or at least 200 founders). For example, the established breeding population can comprise between about 100 and about 200 founders (e.g., about 30-40 female founders and 80-150 male founders) and their descendents of known ancestry. The breeding population typically spans a large number of generations and breeding cycles. For example, an established breeding population can span three, four, five, six, seven, eight, nine or more breeding cycles. The members of the first plant population can thus have the same characteristics. In some embodiments, the members of the first plant population span at least three breeding cycles (e.g., at least four, five, six, seven, eight, or nine breeding cycles). In one class of example embodiments, the first plant population comprises a plurality of inbreds, single cross F1 hybrids, or a combination thereof, the ancestry of each inbred and/or single cross F1 hybrid is known, and each inbred and/or single cross F1 hybrid is a descendent of at least one of three or more founders (e.g., 10, 50, or 100 or more founders). The first population optionally comprises one or more founders, e.g., from which other members of the population are descended.

The first plant population can comprise essentially any number of members. For example, the first plant population optionally comprises between about 50 and about 5000 members (e.g., the first plant population can include 50-5000 inbreds and/or single cross F1 hybrids). As another example, the first plant population can comprise at least about 50, 100, 200, 500, 1000, 2000, 3000, 4000, 5000, or even 6000 or more members. As just one specific example, the first plant population can comprise about 1000 inbreds and between about 3000 and 5000 single cross hybrids.

It is worth noting that the first plant population optionally has any combination of the above characteristics. As just one example, the first plant population can comprise between 50 and 5000 members, including a plurality of inbreds and/or single cross F1 hybrids, each of known ancestry and descended from at least one of three or more founders.

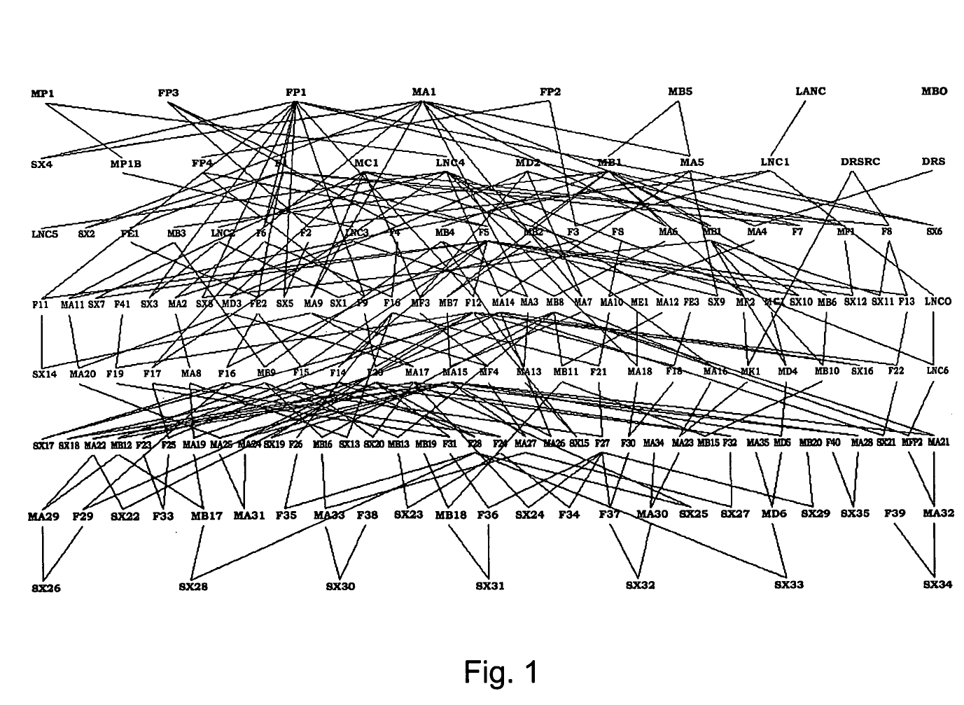

FIG. 1 is a pedigree schematically illustrating the relationships between various inbred lines and single cross hybrids that could, for example, comprise the first plant population. In FIG. 1, SX followed by a number represents a single cross hybrid, while other character combinations designate various inbred lines (except LANC, which represents a population from which inbred line LNC1 was derived). In this figure, the founders include MP1, FP3, FP1, MA1, FP2, MB5, LNC1, and DRS, for example. A line connecting two individuals indicates that one is an ancestor of the other. For example, inbred lines MFP2 and MA21 were crossed to produce, after several generations of selfing, inbred line MA32. (In this example, the line connecting MFP2 and MA32 or MA21 and MA32 represents a distance of one breeding cycle.) As another example, inbred lines F39 and MA32 were crossed to produce single cross F1 hybrid SX34. (In this example, the line connecting F39 and SX34 or MA32 and SX34 represents a distance of less than one breeding cycle.)

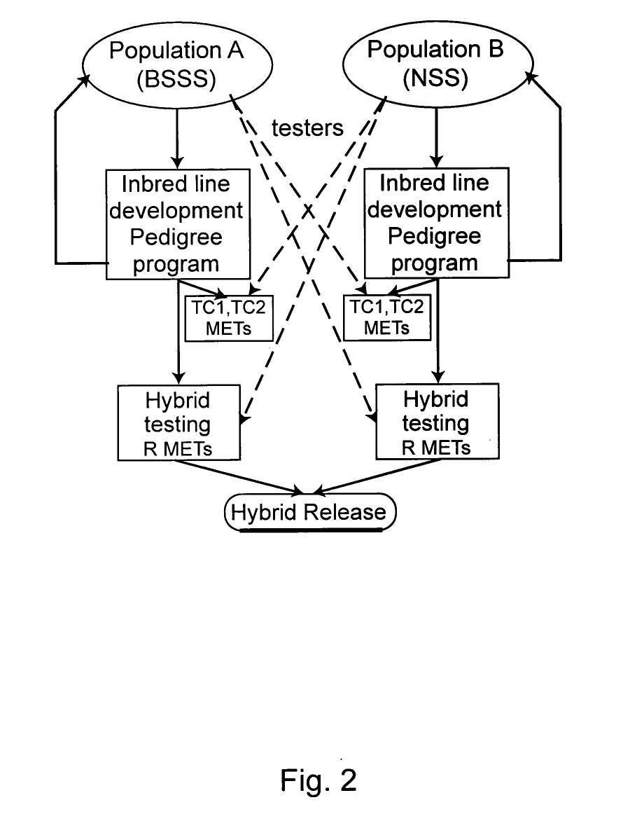

FIG. 2 schematically illustrates an example commercial plant breeding program, for corn in this example. Inbred lines are developed, e.g., from two populations (one male and one female). In a topcross and hybrid testing phase, topcrosses are performed with testers from the opposite population (TC1 and TC2, first and second year topcrosses; MET, multiple environment test).

Typically, the first plant population exhibits variability for the phenotypic trait of interest (e.g., quantitative variability for a quantitative phenotypic trait).

The value of the phenotypic trait in the first plant population is obtained, e.g., by evaluating the phenotypic trait among the members of the first plant population (e.g., quantifying a quantitative phenotypic trait among the members of the population). The phenotype can be evaluated in the members (e.g., the inbreds and/or single cross F1 hybrids) comprising the first plant population. Alternatively, the value of the phenotypic trait in the first plant population can be obtained by evaluating the phenotypic trait among the members of the first plant population in at least one topcross combination with at least one tester parent (e.g., for phenotypic traits which can only be evaluated in hybrids).

The phenotypic trait can be essentially any quantitative or qualitative phenotypic trait, e.g., one of agronomic and/or economic importance. For example, the phenotypic trait can be selected from the group consisting of: yield, grain moisture content, grain oil content, root lodging resistance, stalk lodging resistance, plant height, ear height, disease resistance, insect resistance, drought resistance, grain protein content, test weight, visual or aesthetic appearance, and cob color. These traits, and techniques for evaluating (e.g., quantifying) them, are well known in the art. For example, grain yield is a traditional measure of crop performance. Test weight is a measure of quality. Grain moisture content is important in storage, while root and stalk lodging resistance affect standability and are important during harvest. The methods are similarly applicable to other phenotypic traits, for example, grain phytate content.

The set of genetic markers can comprise essentially any convenient genetic markers. For example, the set of genetic markers can comprise one or more of: a single nucleotide polymorphism (SNP), a multinucleotide polymorphism, an insertion or a deletion of at least one nucleotide (indel), a simple sequence repeat (SSR), a restriction fragment length polymorphism (RFLP), a random amplified polymorphic DNA (RAPD) marker, or an arbitrary fragment length polymorphism (AFLP). As will be evident to one of skill, the number of markers required can vary, e.g., depending on the rate at which linkage disequilibrium declines in the plant species of interest and/or on the type of association analysis performed. The set of genetic markers can include, for example, from 1 to 50,000 markers (e.g., between 1 and 10,000 markers). In one class of embodiments, the set of genetic markers comprises between about 50 and about 2500 markers. For example, the set of genetic markers can comprise at least about 50, 100, 250, 500, 1000, 2000, or even 2500 or more genetic markers. In certain embodiments, the set of genetic markers comprises between one and ten markers (e.g., for candidate gene studies, in which relatively few markers are needed). In other embodiments, the set of genetic markers comprises between 500 and 50,000 markers (e.g., for whole genome scans).

The genotype of the first plant population for the set of genetic markers can be determined experimentally, predicted, or a combination thereof. For example, in one class of embodiments, the genotype of each inbred present in the plant population is experimentally determined and the genotype of each single cross F1 hybrid present in the first plant population is predicted (e.g., from the experimentally determined genotypes of the two inbred parents of each single cross hybrid). Plant genotypes can be experimentally determined by essentially any convenient technique. Many applicable techniques for discovering and/or genotyping genetic markers are known in the art (e.g., those described below in the section entitled “Genetic Markers”). In one preferred class of embodiments, a set of DNA segments from each inbred is sequenced to experimentally determine the genotype of each inbred. Since sequence polymorphisms (e.g., genetic markers) are typically more common in noncoding regions (e.g., introns and untranslated regions), in one class of embodiments the set of DNA segments that is sequenced comprises the 5′-untranslated regions and/or the 3′-untranslated regions of one or more (e.g., two or more) genes. Sequencing techniques (e.g., direct sequencing of PCR amplicons) are well known (see, e.g., Ching et al. (2002) “SNP frequency, haplotype structure and linkage disequilibrium in elite maize inbred lines” BMC Genetics 3: 19).

In some embodiments, a single genetic marker is associated with the phenotypic trait, while in other embodiments, two or more genetic markers (and/or chromosome regions) are associated with the phenotypic trait. Thus, in one class of embodiments, an association between a haplotype comprising two or more genetic markers and the phenotypic trait is provided. The genetic markers comprising a haplotype can be unlinked (e.g., two or more QTL affecting the phenotypic trait can be identified, each of which is associated with one of the markers), or the genetic markers can be physically linked (e.g., the genetic markers can comprise a haplotype block associated with the phenotypic trait, e.g., a SNP haplotype tagged haplotype block).

As noted, the association is evaluated in the first plant population according to a statistical model that incorporates genotypic and phenotypic information about the first plant population. The statistical model typically also exploits relationships among the plants in the first population by incorporating family relationships among the members of the first plant population along with the genetic marker and phenotypic trait data. The model can incorporate family relationships by, for example, including an indication of whether a particular allele is of maternal or paternal origin, or by any other means that permits use of pedigree relationship information to track alleles that are identical by descent in different individuals.

In a preferred class of embodiments, the association between the at least one genetic marker and the phenotypic trait is evaluated by performing Bayesian analysis using a linear model, a mixed linear model, or a nonlinear model. The Bayesian analysis can be implemented, e.g., via a reversible jump Markov chain Monte Carlo algorithm, a delta method, or a profile likelihood algorithm. For example, in one such preferred class of embodiments, the association is evaluated by performing Bayesian analysis using a linear model, the Bayesian analysis being implemented via a reversible jump Markov chain Monte Carlo algorithm. Typically, evaluating the association includes (and/or permits) determining identity by descent information for founder alleles of the at least one genetic marker in one or more pedigrees of related inbreds and hybrids, and permits tracking of the at least one genetic marker throughout such pedigrees. Typically, the Bayesian analysis (e.g., implemented via a reversible jump Markov chain Monte Carlo algorithm) is implemented via a computer program or system.

Bayesian methods, Monte Carlo algorithms, and the like are well known in the art. General references that are useful in understanding relevant concepts include: Gibas and Jambeck (2001) Bioinformatics Computer Skills, O'Reilly, Sebastipol, Calif.; Pevzner (2000) Computational Molecular Biology and Algorithmic Approach, The MIT Press, Cambridge Mass.; Durbin et al. (1998) Biological Sequence Analysis: Probabilistic Models of Proteins and Nucleic Acids, Cambridge University Press, Cambridge, UK; Hinchliffe (1996) Modeling Molecular Structures John Wiley and Sons, NY, N.Y.; and Rashidi and Buehler (2000) Bioinformatic Basics: Applications in Biological Science and Medicine CRC Press LLC, Boca Raton, Fla. Detailed discussions of Monte Carlo statistical analyses are provided in various resources that include, e.g., Robert et al. (1999) Monte Carlo Statistical Methods, Springer-Verlag; Chen et al. (2000) Monte Carlo Methods in Bayesian Computation, Springer-Verlag; Sobol et al. (1994) A Primer for the Monte Carlo Method, CRC Press, LLC; Manno (1999) Introduction to the Monte-Carlo Method, Akademiai Kiado; and Rubinstein (1981) Simulation and the Monte Carlo Method, John Wiley & Sons, Inc. Additional details relating to these statistical methods are found in, e.g., Carlin et al. (1995) “Bayesian model choice via Markov chain Monte Carlo methods” J. Royal Stat. Soc. Series B, 57: 473-84; Carlin et al. (1991) “An iterative Monte Carlo method for nonconjugate Bayesian analysis” Statistics and Computing 1: 119-28; and Pillardy et al. (2001) “Conformation-family Monte Carlo: A new method for crystal structure prediction” Proc. Natl. Acad. Sci. USA 98(22): 12351-6.

In particular, Bayesian methods for QTL mapping (i.e., for evaluating association between a set of genetic markers and a phenotypic trait) are known in the art. For example, Bink et al. (2002) “Multiple QTL mapping in related plant populations via a pedigree-analysis approach” Theor. Appl. Genet. 104: 751-762 and Yi and Xu (2001) “Bayesian mapping of quantitative trait loci under complicated mating designs” Genetics 157: 1759-1771 describe Bayesian analysis implemented via reversible jump Markov chain Monte Carlo algorithms and using linear models, and are hereby incorporated by reference in their entirety. The model presented in Bink et al., for example, incorporates the genotype of two or more plants for a set of genetic markers, values of the phenotypic trait observed in the plants, and family relationships between the plants (by using segregation indicators that indicate maternal or paternal derivation, e.g., of genetic marker and therefore of linked QTL alleles). This model also includes non-genetic factors affecting the trait (e.g., environmental effects).

Bayesian analysis, QTL mapping, and the like are also described in, e.g., Sorensen and Gianola (2002) Likelihood, Bayesian and MCMC methods in quantitative genetics, Springer, N.Y.; Jannink and Fernando (2004) “On the metropolis-hastings acceptance probability to add or drop a quantitative trait locus in markov chain monte carlo-based bayesian analyses” Genetics 166: 641-643; Wu and Jannink (2004) “Optimal sampling of a population to determine QTL location, variance, and allelic number” Theor Appl Genet 108: 1434-42; Jannink (2003) “Selection dynamics and limits under additive-by-additive epistatic gene action” Crop Sci 43: 489-497; Yi and Xu (2000) “Bayesian mapping of quantitative trait loci under the identity-by-descent-based variance component model” Genetics 156: 411-422; Berry et al. (2002) “Assessing probability of ancestry using simple sequence repeat profiles: Applications to maize hybrids and inbreds” Genetics 161: 813-824; Berry et al. (2003) “Assessing probability of ancestry using simple sequence repeat profiles: Applications to maize inbred lines and soybean varieties” Genetics 165: 331-342; and Jannink and Wu (2003) “Estimating allelic number and identity in state of QTLs in interconnected families” Genet Res 81: 133-44. An example software package for Bayesian analysis of QTL in interconnected populations is publicly available at www.public.iastate.edu/˜jjannink/Research/Software.htm.

In another preferred class of embodiments, the association is evaluated by performing a transmission disequilibrium test (see, e.g., the Examples and the references therein). In another class of embodiments, the association is evaluated by a maximum likelihood mixed linear or nonlinear model analysis (see, e.g., Lynch and Walsh (1998) Genetic Analysis of Quantitative Traits, Sinauer Associates, Inc., Sunderland M A, pp 746-755). In yet another class of embodiments, the association is evaluated in the first plant population via an artificial neural network. Such networks are known in the art; see, e.g., Gurney (1999) An Introduction to Neural Networks, UCL Press, 1 Gunpowder Square, London EC4A 3DE, UK; Bishop (1995) Neural Networks for Pattern Recognition, Oxford Univ Press; ISBN: 0198538642; Ripley, Hjort (1995) Pattern Recognition and Neural Networks, Cambridge University Press (Short); and Masters (1993) Practical Neural Network Recipes in C++ (Book&Disk edition) Academic Press.

The target plant population can comprise essentially any number of members that are related and/or unrelated to each other and to the members of the first plant population. The members of the target plant population typically do not themselves comprise the first plant population.

Thus, the target plant population can comprise, e.g., inbred plants, hybrid plants, or a combination thereof. The hybrid plants can comprise, e.g., single cross hybrids, double cross hybrids, hybrid progeny of three-way crosses, or essentially any other hybrids. In a preferred class of embodiments, the target plant population comprises hybrid plants that comprise F1 progeny produced from single crosses between inbred lines. These F1 progeny can be produced, e.g., from single crosses between inbreds comprising the first plant population (where the hybrid plants do not comprise the first plant population), from single crosses between new inbreds that contain preferred alleles (genetic marker and/or QTL alleles) identical by descent or identical by state to those inbreds used in the association mapping analysis, or a combination thereof. Similarly, in one class of embodiments, the target plant population comprises an advanced generation produced from breeding crosses comprising at least one of the members of the first plant population (i.e., the target plant population comprises F2 or later descendants of at least one member of the first plant population).

It is worth noting that the target plant population can comprise actual living plants and/or hypothetical plants (e.g., hypothetical single cross hybrids produced by crossing given pairs of inbred lines of known genetic marker genotype). Typically, if the methods are applied to a hypothetical target plant population, at least one actual plant (e.g., one having the most desirable predicted value of the phenotypic trait) will actually be produced as a living plant.

The genotype of the member(s) of the target plant population for the at least one genetic marker associated with the phenotypic trait can be determined experimentally and/or predicted. Thus, in one class of embodiments, the genotype of the at least one member of the target plant population for the at least one genetic marker is determined experimentally, e.g., by high throughput screening. In another class of embodiments, the genotype of the at least one member of the target plant population for the at least one genetic marker is predicted. For example, the genotype of a single cross F1 hybrid member of the target population can be predicted if the genotypes of its inbred parents are known.

The value of the phenotypic trait in at least one member of the target plant population can be predicted, for example, by a method that incorporates both pedigree and genetic marker information (e.g., both genetic marker genotype and identity by descent and/or identity by state information for genetic marker alleles).

In a preferred class of embodiments, the value of the phenotypic trait in the at least one member of the target plant population is predicted using a best linear unbiased prediction method. Best linear unbiased prediction methods are known in the art; see, e.g., Gianola et al. (2003) “On Marker-Assisted Prediction of Genetic Value: Beyond the Ridge” Genetics 163: 347-365 and Bink et al. (2002) “Multiple QTL mapping in related plant populations via a pedigree-analysis approach” Theor. Appl. Genet. 104: 751-762. Alternatively, other methods can be used to predict the value of the phenotypic trait in the at least one member of the target plant population, e.g., a multiple regression method, a selection index technique, a ridge regression method, a linear optimization method, or a non-linear optimization method. Such methods are well known; see, e.g., Johnson, B. E. et al. (1988) “A model for determining weights of traits in simultaneous multitrait selection” Crop Sci. 28: 723-728.

The first and target plant populations can comprise essentially any type of plants. For example, in a preferred class of embodiments, the first and target plant populations comprise (e.g., consist of) diploid plants. As noted previously, the methods are particularly applicable to hybrid crop plants. Thus, in preferred embodiments, the first and target plant populations are selected from the group consisting of: maize (e.g., Zea mays), soybean, sorghum, wheat, sunflower, rice, canola, cotton, and millet.

A QTL identified by the methods herein (e.g., a QTL allele linked to the at least one genetic marker associated with the phenotypic trait) can optionally be cloned and expressed, e.g., to create a transgenic plant having a desirable value of the phenotypic trait. Thus, in one class of embodiments, the methods include cloning a gene that is linked to the at least one genetic marker associated with the phenotypic trait, wherein expression of the gene affects the phenotypic trait. The methods optionally also include constructing a transgenic plant by expressing the cloned gene in a host plant.

Digital Systems

In general, various automated systems can be used to perform some or all of the method steps as noted herein. In addition to practicing some or all of the method steps herein, digital or analog systems, e.g., comprising a digital or analog computer, can also control a variety of other functions such as a user viewable display (e.g., to permit viewing of method results by a user) and/or control of output features (e.g., to assist in marker assisted selection or control of automated field equipment).

For example, certain of the methods described above are optionally (and typically) implemented via a computer program or programs (e.g., that perform or assist in performing a transmission disequilibrium test, Bayesian analysis and/or phenotype prediction). Thus, the present invention provides digital systems, e.g., computers, computer readable media, and/or integrated systems comprising instructions (e.g., embodied in appropriate software) for performing the methods herein. For example, a digital system comprising instructions for evaluating an association in the first plant population between at least one genetic marker and a phenotypic trait and for predicting the value of the phenotypic trait in at least one member of a second, target plant population, as described herein, is a feature of the invention. The digital system can also include information (data) corresponding to plant genotypes for a set of genetic markers, phenotypic values, and/or family relationships. The system can also aid a user in performing marker assisted selection according to the methods herein, or can control field equipment which automates selection, harvesting, and/or breeding schemes.

Standard desktop applications such as word processing software (e.g., Microsoft Word™ or Corel WordPerfect™) and/or database software (e.g., spreadsheet software such as Microsoft Excel™, Corel Quattro Pro™, or database programs such as Microsoft Access™ or Paradox™) can be adapted to the present invention by inputting data which is loaded into the memory of a digital system, and performing an operation as noted herein on the data. For example, systems can include the foregoing software having the appropriate pedigree data, phenotypic information, associations between phenotype and pedigree, etc., e.g., used in conjunction with a user interface (e.g., a GUI in a standard operating system such as a Windows, Macintosh or LINUX system) to perform any analysis noted herein, or simply to acquire data (e.g., in a spreadsheet) to be used in the methods herein.

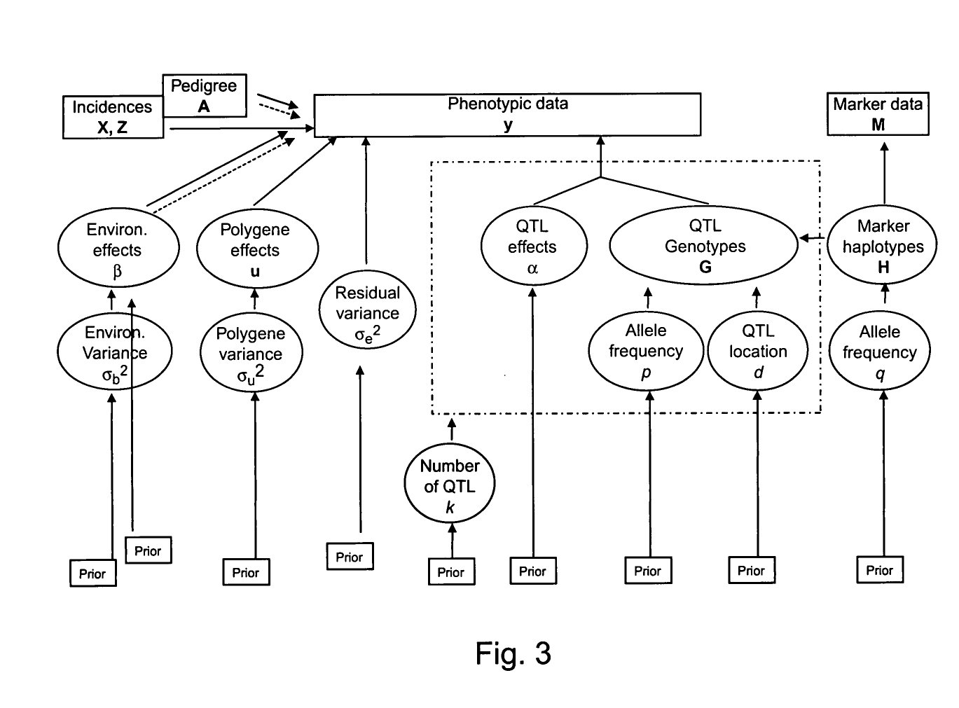

Software for performing statistical analysis can also be included in the digital system. For example, Bayesian analysis can be performed using software such as that described in Bink et al. (2002) “Multiple QTL mapping in related plant populations via a pedigree-analysis approach” Theor. Appl. Genet. 104: 751-762, or a modified version thereof. FIG. 3 schematically depicts a software implementation of this Bayesian analysis of QTLs in a complex pedigree.

Systems typically include, e.g., a digital computer with software for performing association analysis and/or phenotypic value prediction, or for performing Bayesian analysis, e.g., implemented via a reversible jump Markov chain Monte Carlo algorithm, or the like, as well as data sets entered into the software system comprising plant genotypes for a set of genetic markers, phenotypic values, family relationships, and/or the like. The computer can be, e.g., a PC (Intel x86 or Pentium chip-compatible DOS,™ OS2,™ WINDOWS,™ WINDOWS NT,™ WINDOWS95,™ WINDOWS98,™ LINUX, Apple-compatible, MACINTOSH™ compatible, Power PC compatible, or a UNIX compatible (e.g., SUN™ work station) machine) or other commercially common computer which is known to one of skill. Software for performing association analysis and/or phenotypic value prediction can be constructed by one of skill using a standard programming language such as Visualbasic, Fortran, Basic, Java, or the like, according to the methods herein.

Any system controller or computer optionally includes a monitor which can include, e.g., a cathode ray tube (“CRT”) display, a flat panel display (e.g., active matrix liquid crystal display, liquid crystal display), or others. Computer circuitry is often placed in a box which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user and for user selection of genetic marker genotype, phenotypic value, or the like in the relevant computer system.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the system to carry out any desired operation. For example, in addition to performing statistical analysis, a digital system can instruct selection of plants comprising certain markers, or control field machinery for harvesting, selecting, crossing or preserving crops according to the relevant method herein.

The invention can also be embodied within the circuitry of an application specific integrated circuit (ASIC) or programmable logic device (PLD). In such a case, the invention is embodied in a computer readable descriptor language that can be used to create an ASIC or PLD. The invention can also be embodied within the circuitry or logic processors of a variety of other digital apparatus, such as PDAs, laptop computer systems, displays, image editing equipment, etc.

Identifying New Allelic Variants

The present invention also provides methods that can be used to identify new allelic variants of a QTL affecting a phenotypic trait. Association analysis can be performed to identify at least one genetic marker associated with the phenotypic trait. Novel alleles of the genetic marker, and thus possibly of a QTL associated with the genetic marker, can be identified in non-adapted germplasm. Such novel allelic variants can then, e.g., be bred into the adapted germplasm (e.g., a commercial breeding population).

Thus, one general class of embodiments provides methods of selecting a plant. In the methods, an association between at least one genetic marker and the phenotypic trait is provided. The association is evaluated in a first plant population, which first plant population is an established breeding population or a portion thereof. The association is evaluated in the first plant population according to a statistical model that incorporates a genotype of the first plant population for a set of genetic markers and a value of the phenotypic trait in the first plant population. The statistical model can also incorporate family relationships among the members of the first plant population. One or more plants from one or more non-adapted lines are then provided. The one or more plants are selected for a selected genotype comprising the at least one genetic marker associated with the phenotypic trait. The selected genotype can comprise, e.g., at least one allele of at least one of the genetic markers associated with the phenotypic trait that is novel with respect to the genetic marker alleles found in the first population. The genotype of the one or more plants for the at least one genetic marker is typically determined experimentally, by any convenient technique.

A novel genetic marker genotype can indicate the presence of a novel allele of a QTL associated with the genetic marker (and with the phenotypic trait). To determine if this putative novel QTL allele is one that favorably affects the phenotypic trait, the methods can include evaluating the phenotypic trait (e.g., quantifying a quantitative phenotypic trait) in the one or more plants having the selected genotype. At least one plant having the selected genotype and a desirable value of the phenotypic trait can be selected. In addition, the at least one selected plant having the selected genotype and the desirable value of the phenotypic trait can be bred with at least one other plant (e.g., to introduce the genetic marker allele and thus the putative novel QTL allele into the adapted germplasm).

The first plant population typically comprises a plurality of inbreds, single cross F1 hybrids, or a combination thereof. For example, in one class of embodiments, the first plant population comprises a plurality of inbreds. In another class of embodiments, the first plant population comprises a plurality of single cross F1 hybrids. In yet another class of embodiments, the first plant population comprises a plurality of a combination of inbreds and single cross F1 hybrids. The first plant population optionally consists of inbreds, single cross F1 hybrids, or a combination thereof. The inbreds can be related and/or unrelated to each other, and the single cross F1 hybrids can be produced from single crosses of said inbred lines and/or one or more additional inbred lines.

As noted, the members of the first plant population are sampled from an established breeding population (e.g., a commercial breeding population). FIG. 1 is a pedigree schematically illustrating the relationships between various inbred lines and single cross hybrids that could, for example, comprise the first plant population. Characteristics of established breeding populations and/or first plant populations noted for the embodiments described above apply to these embodiments as well. Thus, for example, in one class of embodiments, the first plant population comprises a plurality of inbreds, single cross F1 hybrids, or a combination thereof, the ancestry of each inbred and/or single cross F1 hybrid is known, and each inbred and/or single cross F1 hybrid is a descendent of at least one of three or more founders (e.g., 10, 50, or 100 or more founders). Similarly, in some embodiments, the members of the first plant population span at least three breeding cycles (e.g., at least four, five, six, seven, eight, or nine breeding cycles). In one class of embodiments, the established breeding population comprises at least three founders and their descendents (e.g., at least 10 founders, at least 50 founders, at least 100 founders, or at least 200 founders, e.g., between about 100 and about 200 founders and their descendents), where the ancestry of the descendents is known. The established breeding population can span, e.g., three, four, five, six, seven, eight, nine or more breeding cycles.

The first plant population can comprise essentially any number of members. For example, the first plant population optionally comprises between about 50 and about 5000 members (e.g., the first plant population can include 50-5000 inbreds and/or single cross F1 hybrids). As another example, the first plant population can comprise at least about 50, 100, 200, 500, 1000, 2000, 3000, 4000, 5000, or even 6000 or more members.

It is worth noting that the first plant population optionally has any combination of the above characteristics. As just one example, the first plant population can comprise between 50 and 5000 members, including a plurality of inbreds and/or single cross F1 hybrids, each of known ancestry and descended from at least one of three or more founders.

The phenotypic trait can be a quantitative trait, e.g., for which a quantitative value can be provided. Alternatively, the phenotypic trait can be a qualitative trait, e.g., for which a qualitative value can be provided. The trait can be determined by a single gene, or it can be determined by two or more genes.

Typically, the first plant population exhibits variability for the phenotypic trait of interest (e.g., quantitative variability for a quantitative phenotypic trait).

The value of the phenotypic trait in the first plant population is obtained, e.g., by evaluating the phenotypic trait among the members of the first plant population (e.g., quantifying a quantitative trait). The phenotype can be evaluated in the plants (e.g., the inbreds and/or single cross hybrids) comprising the first plant population. Alternatively, the value of the phenotypic trait in the first plant population can be obtained by evaluating the phenotypic trait among the members of the first plant population in at least one topcross combination with at least one tester parent, and optionally calculating Best Linear Unbiased Predictors of the phenotype for the genotype of interest.

The phenotypic trait can be essentially any qualitative or quantitative phenotypic trait, e.g., one of agronomic and/or economic importance. For example, the phenotypic trait can be selected from the group consisting of: yield, grain moisture content, grain oil content, root lodging resistance, stalk lodging resistance, plant height, ear height, disease resistance, insect resistance, drought resistance, grain protein content, test weight, visual and/or aesthetic appearance, and cob color. These traits, and techniques for quantifying them, are well known in the art. For example, grain yield is a traditional measure of crop performance. Test weight is a measure of quality. Grain moisture content is important in storage, while root and stalk lodging resistance affect standability and are important during harvest. The methods are similarly applicable to other phenotypic traits, for example, grain phytate content.

The set of genetic markers can comprise essentially any convenient genetic markers. For example, the set of genetic markers can comprise one or more of: a single nucleotide polymorphism (SNP), a multinucleotide polymorphism, an insertion or a deletion of at least one nucleotide (indel), a simple sequence repeat (SSR), a restriction fragment length polymorphism (RFLP), an EST sequence or a unique nucleotide sequence of 20-40 bases used as a probe (oligonucleotides), a random amplified polymorphic DNA (RAPD) marker, or an arbitrary fragment length polymorphism (AFLP). As will be evident to one of skill, the number of markers required can vary, e.g., depending on the rate at which linkage disequilibrium declines in the plant species of interest and/or on the type of association analysis performed. The set of genetic markers can include, for example, from 1 to 50,000 markers (e.g., between 1 and 10,000 markers). In one class of embodiments, the set of genetic markers comprises between about 50 and about 2500 markers. For example, the set of genetic markers can comprise at least about 50, 100, 250, 500, 1000, 2000, or even 2500 or more genetic markers. In certain embodiments, the set of genetic markers comprises between one and ten markers (e.g., for candidate gene studies, in which relatively few markers are needed). In other embodiments, the set of genetic markers comprises between 500 and 50,000 markers (e.g., for whole genome scans).

The genotype of the first plant population for the set of genetic markers can be determined experimentally, predicted, or a combination thereof. For example, in one class of embodiments, the genotype of each inbred present in the first plant population is experimentally determined and the genotype of each F1 hybrid present in the first plant population is predicted (e.g., from the experimentally determined genotypes of the two inbred parents of each single cross hybrid). Plant genotypes can be experimentally determined by essentially any convenient technique. Many applicable techniques for discovering and/or genotyping genetic markers are known in the art (e.g., those described below in the section entitled “Genetic Markers”). In one preferred class of embodiments, a set of DNA segments from each inbred is sequenced to experimentally determine the genotype of each inbred. Since sequence polymorphisms (e.g., genetic markers) are typically more common in noncoding regions (e.g., introns and untranslated regions), in one class of embodiments the set of DNA segments that is sequenced comprises the 5′-untranslated regions and/or the 3′-untranslated regions of one or more (e.g., two or more) genes. As noted above, sequencing techniques (e.g., direct sequencing of PCR amplicons) are well known.

In some embodiments, a single genetic marker is associated with the phenotypic trait, while in other embodiments, two or more genetic markers are associated with the phenotypic trait. Thus, in one class of embodiments, an association between a haplotype comprising two or more genetic markers and the phenotypic trait is provided. The genetic markers comprising a haplotype can be unlinked (e.g., two or more QTL affecting the phenotypic trait can be identified, each of which is associated with one of the markers), or the genetic markers can be physically linked (e.g., the genetic markers can comprise a haplotype block associated with the phenotypic trait, e.g., a SNP haplotype tagged haplotype block).

In a preferred class of embodiments, the association between the at least one genetic marker and the phenotypic trait is evaluated by performing Bayesian analysis using a linear model, a mixed linear model, or a nonlinear model. The Bayesian analysis can be implemented, e.g., via a reversible jump Markov chain Monte Carlo algorithm, a delta method, or a profile likelihood algorithm. For example, in one such preferred class of embodiments, the association is evaluated by performing Bayesian analysis using a linear model, the Bayesian analysis being implemented via a reversible jump Markov chain Monte Carlo algorithm. Typically, the Bayesian analysis (e.g., implemented via a reversible jump Markov chain Monte Carlo algorithm) is implemented via a computer program or system.

As noted above, Bayesian methods, Monte Carlo algorithms, and the like are well known in the art. In particular, Bayesian methods for QTL mapping (i.e., for evaluating association between a set of genetic markers and a phenotypic trait) are known; see, e.g., Bink et al. and Yi and Xu, both supra.

In another preferred class of embodiments, the association is evaluated by performing a transmission disequilibrium test. In another class of embodiments, the association is evaluated by a maximum likelihood mixed linear or nonlinear model analysis. In yet another class of embodiments, the association is evaluated in the first plant population via an artificial neural network. As noted, such networks are known in the art; see, e.g., the references above.

The first plant population and the one or more non-adapted lines can comprise essentially any type of plants. For example, in a preferred class of embodiments, the first plant population and the one or more non-adapted lines comprise (e.g., consist of) diploid plants. In preferred embodiments, the first plant population and the one or more non-adapted lines are selected from the group consisting of: maize (e.g., Zea mays), soybean, sorghum, wheat, sunflower, rice, canola, cotton, and millet.

A QTL identified by the methods herein (e.g., a QTL allele linked to the at least one genetic marker associated with the phenotypic trait) can optionally be cloned and expressed, e.g., to create a transgenic plant having a desirable value of the phenotypic trait. Thus, in one class of embodiments, the methods include cloning a gene that is linked to the at least one genetic marker associated with the phenotypic trait from the at least one selected plant having the selected genotype and the desirable value of the phenotypic trait, wherein expression of the gene affects the phenotypic trait (i.e., cloning the novel QTL allele from the non-adapted plant). The methods optionally also include constructing a transgenic plant by expressing the cloned gene in a host plant.

All of the various optional configurations and features noted for the embodiments above apply here as well, to the extent they are relevant.

Plants

Plants selected, provided, or produced by any of the methods herein form another feature of the invention, as do transgenic plants created by any of the methods herein.

Genetic Markers

In the following discussion, the phrase “nucleic acid,” “polynucleotide,” “polynucleotide sequence” or “nucleic acid sequence” refers to deoxyribonucleotides or ribonucleotides and polymers thereof in either single- or double-stranded form. Unless specifically stated, the term encompasses nucleic acids containing known analogs of natural nucleotides which have similar binding properties as the reference nucleic acid.

The ability to characterize an individual by its genome is due to the inherent variability of genetic information. Typically, genetic markers are polymorphic regions of a genome and the complementary oligonucleotides which bind to these regions. Polymorphic sites are often located in noncoding regions of DNA (e.g., 5′ or 3′ untranslated regions, intergenic regions, and the like). Polymorphic sites are also found in coding regions, where, for example, a nucleotide change can be silent and not result in amino acid substitution in the encoded protein, result in conservative amino acid substitution, or result in nonconservative amino acid substitution. As would be expected, polymorphic sites (particularly insertions, deletions, and nucleotide changes resulting in nonconservative substitutions) are relatively uncommon in regions coding for proteins whose function is essential. Typically, the presence or absence of a particular genetic marker identifies individuals by their unique nucleic acid sequence; in other instances, a genetic marker is found in all individuals but the individual is identified by where, in the genome, the genetic marker is located.

The major causes of genetic variability, and thus the major sources of genetic markers, are insertions (additions), deletions, nucleotide substitutions (point mutations), recombination events, and transposable elements within the genome of individuals in a plant population. As one example, point mutations can result from errors in DNA replication or damage to the DNA. As another example, insertions and deletions can result from inaccurate recombination events. As yet another example, variability can arise from the insertion or excision of a transposable element (a DNA sequence that has the ability to move or to jump to new locations with the genome, autonomously or non-autonomously).

The net result of such heritable changes in DNA sequences is that individuals have different sequences. Regions comprising polymorphic sites (sites where DNA sequences are different among individuals or between the two chromosomes in a given individual) can be used as genetic markers.

Genetic markers can be classified by the type of change (e.g., insertion or deletion of one or more nucleotides or substitution of one or more nucleotides) and/or by the way in which the change is detected (e.g., a RFLP and an AFLP can each result from insertion, deletion, or substitution).

Discovery, detection, and genotyping of various genetic markers has been well described in the literature. See, e.g., Henry, ed. (2001) Plant Genotyping. The DNA Fingerprinting of Plants Wallingford: CABI Publishing; Phillips and Vasil, eds. (2001) DNA-based Markers in Plants Dordrecht: Kluwer Academic Publishers; Pejic et al. (1998) “Comparative analysis of genetic similarity among maize inbred lines detected by RFLPs, RAPDs, SSRs and AFLPs” Theor. App. Genet. 97: 1248-1255; Bhattramakki et al. (2002) “Insertion-deletion polymorphisms in 3′ regions of maize genes occur frequently and can be used as highly informative genetic markers” Plant Mol. Biol. 48: 539-47; Nickerson et al. (1997) “PolyPhred: automating the detection and genotyping of single nucleotide substitutions using fluorescence-based resequencing” Nucleic Acids Res. 25: 2745-2751; Underhill et al. (1997) “Detection of numerous Y chromosome biallelic polymorphisms by denaturing high-performance liquid chromatography” Genome Res. 7: 996-1005; Shi (2001) “Enabling large-scale pharmacogenetic studies by high-throughput mutation detection and genotyping technologies” Clin. Chem. 47: 164-172; Kwok (2000) “High-throughput genotyping assay approaches” Pharmacogenomics 1: 95-100; Rafalski et al. (2002) “The genetic diversity of components of rye hybrids” Cell Mol Biol Lett 7: 471-5; Ching and Rafalski (2002) “Rapid genetic mapping of ests using SNP pyrosequencing and indel analysis” Cell Mol Biol Lett. 7: 803-10; and Powell et al. (1996) “The comparison of RFLP, RAPD, AFLP and SSR (microsatellite) markers for germplasm analysis” Mol. Breeding 2: 225-238.

SNPs

Sites in the DNA sequence where individuals differ at a single DNA base are called single nucleotide polymorphisms (SNPs). A SNP can result, e.g., from a point mutation.

SNPs can be discovered by any of a number of techniques known in the art. For example, SNPs can be detected by direct sequencing of DNA segments, e.g., amplified by PCR, from several individuals (see, e.g., Ching et al. (2002) “SNP frequency, haplotype structure and linkage disequilibrium in elite maize inbred lines” BMC Genetics 3: 19). As another example, SNPs can be discovered by computer analysis of available sequences (e.g., ESTs, STSS) derived from multiple genotypes (see, e.g., Marth et al. (1999) “A general approach to single-nucleotide polymorphism discovery” Nature Genetics 23: 452-456 and Beutow et al. (1999) “Reliable identification of large numbers of candidate SNPs from public EST data” Nature Genetics 21: 323-325). (Indels, insertions or deletions of one or more nucleotides, can also be discovered by sequencing and/or computer analysis, e.g., simultaneously with SNP discovery.)

Similarly, SNPs can be genotyped by sequencing. SNPs can also be genotyped by various other methods (including high throughput methods) known in the art, for example, using DNA chips, allele-specific hybridization, allele-specific PCR, and primer extension techniques. See, e.g., Lindblad-Toh et al. (2000) “Large-scale discovery and genotyping of single-nucleotide polymorphisms in the mouse” Nature Genetics 24: 381-386; Bhattramakki and Rafalski (2001) “Discovery and application of single nucleotide polymorphism markers in plants” in Plant Genotyping: The DNA Fingerprinting of Plants, CABI Publishing; Syvanen (2001) “Accessing genetic variation: genotyping single nucleotide polymorphisms” Nat. Rev. Genet. 2: 930-942; Kuklin et al. (1998) “Detection of single-nucleotide polymorphisms with the WAVE TM DNA fragment analysis system” Genetic Testing 1: 201-206; Gut (2001) “Automation in genotyping single nucleotide polymorphisms” Hum. Mutat. 17: 475-492; Lemieux (2001) “Plant genotyping based on analysis of single nucleotide polymorphisms using microarrays” in Plant Genotyping: The DNA Fingerprinting of Plants, CABI Publishing; Edwards and Mogg (2001) “Plant genotyping by analysis of single nucleotide polymorphisms” in Plant Genotyping: The DNA Fingerprinting of Plants, CABI Publishing; Ahmadian et al. (2000) “Single-nucleotide polymorphism analysis by pyrosequencing” Anal. Biochem. 280: 103-110; Useche et al. (2001) “High-throughput identification, database storage and analysis of SNPs in EST sequences” Genome Inform Ser Workshop Genome Inform 12: 194-203; Pastinen et al. (2000) “A system for specific, high-throughput genotyping by allele-specific primer extension on microarrays” Genome Res. 10: 1031-1042; Hacia (1999) “Determination of ancestral alleles for human single-nucleotide polymorphisms using high-density oligonucleotide arrays” Nature Genet. 22: 164-167; and Chen et al. (2000) “Microsphere-based assay for single-nucleotide polymorphism analysis using single base chain extension” Genome Res. 10: 549-557.

Multinucleotide polymorphisms can be discovered and detected by analogous methods.

RFLPs

As noted above, different individuals have different genomic DNA sequences. Thus, when these DNA sequences are digested with one or more restriction endonucleases that recognize specific restriction sites, some of the resulting fragments are of different lengths. The resulting fragments are restriction fragment length polymorphisms.

The phrase restriction fragment length polymorphisms or RFLPs refers to inherited differences in restriction enzyme sites (for example, caused by base changes in the target site) or additions or deletions in regions flanked by the restriction enzyme sites that result in differences in the lengths of the fragments produced by cleavage with a relevant restriction enzyme. A point mutation leads to either longer fragments if the mutation is within the restriction site or shorter fragments if the mutation creates a restriction site. Insertions and transposable element integration lead to longer fragments, and deletions lead to shorter fragments.

Originally, RFLP analysis was performed by Southern blot and hybridization. RFLP analysis is currently more typically performed by PCR. A pair of oligonucleotide primers linking the region comprising the RFLP is used to amplify a fragment from genomic DNA. The size of the PCR products can be analyzed directly, and if the fragment contains a polymorphic restriction site, the PCR products can be digested with the enzyme and the size of the digested products can be analyzed.

Techniques for discovery and genotyping of RFLPs have been well described in the literature. See, for example, Gauthier et al. (2002) “RFLP diversity and relationships among traditional European maize populations” Theor. Appl. Genet. 105: 91-99; Ramalingam et al. (2003) “Candidate defense genes from rice, barley, and maize and their association with qualitative and quantitative resistance in rice” Mol Plant Microbe Interact 16: 14-24; Guo et al. (2002) “Restriction fragment length polymorphism assessment of the heterogeneous nature of maize population GT-MAS:gk and field evaluation of resistance to aflatoxin production by Aspergillus flavus” J Food Prot 65: 167-71; Pejic et al. (1998) “Comparative analysis of genetic similarity among maize inbred lines detected by RFLPs, RAPDs, SSRs and AFLPs” Theor. App. Genet. 97: 1248-1255; and Powell et al. (1996) “The comparison of RFLP, RAPD, AFLP and SSR (microsatellite) markers for germplasm analysis” Mol. Breeding 2: 225-238.

RAPDs

To identify a Random Amplified Polymorphic DNA (RAPD) marker, an oligonucleotide (e.g., an octanucleotide, a decanucleotide) is randomly chosen. The complexity of plant genomic DNA is high enough that a pair of sites complementary to the oligonucleotide may by chance exist in the correct orientation and close enough together to permit PCR amplification of a fragment bounded by the pair of sites. With some randomly chosen oligonucleotides, no sequences are amplified. With other oligonucleotides, products of the same length are generated from genomic DNA of different individuals. With yet other oligonucleotides, however, product lengths are not the same for every individual in a population, providing a useful RAPD marker. RAPD markers have been described in, e.g., Pejic et al. (1998) “Comparative analysis of genetic similarity among maize inbred lines detected by RFLPs, RAPDs, SSRs and AFLPs” Theor. App. Genet. 97: 1248-1255; and Powell et al. (1996) “The comparison of RFLP, RAPD, AFLP and SSR (microsatellite) markers for germplasm analysis” Mol. Breeding 2: 225-238.

AFLPs

Arbitrary fragment length polymorphisms (AFLPs) can also be used as genetic markers (Vos, P., et al., Nucl. Acids Res. 23: 4407 (1995)). The phrase “arbitrary fragment length polymorphism” refers to selected restriction fragments which are amplified before or after cleavage by a restriction endonuclease. The amplification step allows easier detection of specific restriction fragments rather than determining the size of all restriction fragments and comparing the sizes to a known control.

AFLP allows the detection of a large number of polymorphic markers (see, supra) and has been used for genetic mapping of plants (Becker et al. (1995) Mol. Gen. Genet. 249: 65; and Meksem et al. (1995) Mol. Gen. Genet. 249: 74) and to distinguish among closely related bacteria species (Huys et al. (1996) Int'l J. Systematic Bacteriol. 46: 572).

SSRs

Simple sequence repeats (SSRs) are short tandem repeats (e.g., di-, tri- or tetra-nucleotide tandem repeats). SSRs can occur at high levels within a genome. For example, dinucleotide repeats have been reported to occur in the human genome as many as 50,000 times, with n (the number of times the dinucleotide sequence is tandemly repeated within a given SSR region) varying from 10 to 60 (Jacob et al. (1991) Cell 67: 213). SSRs have also been found in higher plants; see, e.g., Taramino and Tingey (1996) “Simple sequence repeats for germplasm analysis and mapping in maize” Genome 39: 277-287; Condit and Hubbell (1991) Genome 34: 66; Peakall et al. (1998) “Cross-species amplification of soybean (Glycine max) simple sequence repeats (SSRs) within the genus and other legume genera: implications for the transferability of SSRs in plants” Mol Biol Evol 15: 1275-87; Morgante et al. (1994) “Genetic mapping and variability of seven soybean simple sequence repeat loci” Genome 37: 763-9; and Zietkiewicz et al. (1994) “Genome fingerprinting by simple sequence repeat (SSR)-anchored polymerase chain reaction amplification” Genomics 20: 176-83.

Briefly, SSR data can be generated, e.g., by hybridizing primers to conserved regions of the plant genome which flank an SSR region. PCR is then used to amplify the nucleotide repeats between the primers. The amplified sequences are then electrophoresed to determine the size of the amplified fragment and therefore the number of di-, tri- and tetra-nucleotide repeats.

Other Markers

Other genetic markers and methods of detecting sequence polymorphisms are known in the art and can be applied to the practice of the present invention, including, but not limited to, single-stranded conformation polymorphisms (SSCPs), amplified variable sequences, isozyme markers, allele-specific hybridization, and self-sustained sequence replication. See, e.g., Orita et al. (1989) “Detection of polymorphisms of human DNA by gel electrophoresis as single-strand conformation polymorphisms” Proc. Natl. Acad. Sci. USA 86: 2766-2770; U.S. Pat. No. 6,399,855 to Beavis, entitled “QTL mapping in plant breeding populations”; and the references above. Candidate genes identified in other studies, e.g., gene function studies, studies of biochemical pathways affecting the phenotypes of interest, physiology of the traits of interest, and the like, can also be used as markers in the first population and the target population.

Haplotype Blocks

Sets of nearby genetic markers on a given chromosome can be inherited in blocks. In some situations, the haplotype of such a block (e.g., a haplotype tag, e.g., comprising the haplotype of a few SNPs representative of a greater number of polymorphisms in a block) may be more informative than the haplotype of a single genetic marker within the block (e.g., a single SNP). See, e.g., the description of haplotype tags in Rafalski (2002) “Applications of single nucleotide polymorphisms in crop genetics” Curr. Opin. Plant Bio. 5: 94-100 and Johnson et (2001) “Haplotype tagging for the identification of common disease genes” Nat. Genet. 29: 233-237.

Molecular Biological Techniques

In practicing the present invention, many conventional techniques in molecular biology and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif. (“Berger”); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2004) (“Ausubel”)). Other useful references for cell isolation and culture (e.g., for subsequent nucleic acid isolation) include, e.g., Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg N.Y.) and Atlas and Parks (Eds.) The Handbook of Microbiological Media (1993) CRC Press, Boca Raton, Fla.

Oligonucleotides (e.g., for use as PCR primers, for use in genetic marker detection methods, or the like) can be obtained by a number of well known techniques. For example, oligonucleotides can be synthesized chemically according to the solid phase phosphoramidite triester method described by Beaucage and Caruthers (1981), Tetrahedron Letts., 22(20): 1859-1862, e.g., using a commercially available automated synthesizer, e.g., as described in Needham-VanDevanter et al. (1984) Nucleic Acids Res., 12: 6159-6168. Oligonucleotides (including, e.g., labeled or modified oligos) can also be ordered from a variety of commercial sources known to persons of skill. There are many commercial providers of oligo synthesis services, and thus, this is a broadly accessible technology. Any nucleic acid can be custom ordered from any of a variety of commercial sources, such as The Midland Certified Reagent Company (www.mcrc.com), The Great American Gene Company (www.genco.com), ExpressGen Inc. (www.expressgen.com), QIAGEN (http://oligos.qiagen.com) and many others.

Positional Cloning

Positional gene cloning uses the proximity of at least one genetic marker to physically define a cloned chromosomal fragment that is linked to a QTL identified using the statistical methods herein. Clones of such linked nucleic acids have a variety of uses, including as genetic markers for identification of linked QTLs in subsequent marker assisted selection protocols, and to improve desired properties in recombinant plants where expression of the cloned sequences in a transgenic plant affects the phenotypic trait of interest. Common linked sequences which are desirably cloned include open reading frames, e.g., encoding proteins which provide a molecular basis for an observed QTL. If one or more markers are proximal to an open reading frame, they may hybridize to a given DNA clone, thereby identifying a clone on which the open reading frame is located. If flanking markers are more distant, a fragment containing the open reading frame may be identified by constructing a contig of overlapping clones.

In certain applications, it is advantageous to make or clone large nucleic acids to identify nucleic acids more distantly linked to a given marker, or isolate nucleic acids linked to or responsible for QTLs as identified herein. It will be appreciated that a nucleic acid genetically linked to a polymorphic nucleotide optionally resides up to about 50 centimorgans from the polymorphic nucleic acid, although the precise distance will vary depending on the cross-over frequency of the particular chromosomal region. Typical distances from a polymorphic nucleotide are in the range of 1-50 centimorgans, for example, often less than 1 centimorgan, less than about 1-5 centimorgans, about 1-5, 1, 5, 10, 15, 20, 25, 30, 35, 40, 45 or 50 centimorgans, etc.

Many methods of making large recombinant RNA and DNA nucleic acids, including recombinant plasmids, recombinant lambda phage, cosmids, yeast artificial chromosomes (YACs), P1 artificial chromosomes, bacterial artificial chromosomes (BACs), and the like are known. A general introduction to YACs, BACs, PACs and MACs as artificial chromosomes is described in Monaco & Larin (1994) Trends Biotechnol. 12: 280-286. Examples of appropriate cloning techniques for making large nucleic acids, and instructions sufficient to direct persons of skill through many cloning exercises are also found in Berger, Sambrook, and Ausubel, all supra.

In one aspect; nucleic acids hybridizing to the genetic markers linked to QTLs identified by the above methods are cloned into large nucleic acids such as YACs, or are detected in YAC genomic libraries cloned from the crop of choice. The construction of YACs and YAC libraries is known. See, e.g., Berger (supra), Ausubel (supra), Burke et al. (1987) Science 236: 806-812, Anand et al. (1989) Nucleic Acids Res. 17: 3425-3433, Anand et al. (1990) Nucleic Acids Res. 18: 1951-1956, and Riley (1990) Nucleic Acids Res. 18: 2887-2890. YAC libraries containing large fragments of soybean DNA have been constructed (see Funke & Kolchinsky (1994) CRC Press, Boca Raton, Fla. pp. 125-308; Marek & Shoemaker (1996) Soybean Genet. Newsl. 23: 126-129; Danish et al. (1997) Soybean Genet. Newsl. 24: 196-198). YAC libraries for many other commercially important crops are available or can be constructed using known techniques.

Similarly, cosmids or other molecular vectors such as BAC and P1 constructs are also useful for isolating or cloning nucleic acids linked to genetic markers. Cosmid cloning is also known. See, e.g., Ausubel; Ish-Horowitz & Burke (1981) Nucleic Acids Res. 9: 2989-2998; Murray (1983) LAMBDA II (Hendrix et al., eds.) pp. 395432, Cold Spring Harbor Laboratory, N.Y.; Frischauf et al. (1983) J. Mol. Biol. 170: 827-842; and Dunn & Blattner (1987) Nucleic Acids Res. 15: 2677-2698, and the references cited therein. Construction of BAC and P1 libraries is known; see, e.g., Ashworth et al. (1995) Anal. Biochem. 224: 564-571; Wang et al. (1994) Genomics 24(3): 527-534; Kim et al. (1994) Genomics 22: 336-9; Rouquier et al. (1994) Anal. Biochem. 217: 205-9; Shizuya et al. (1992) Proc. Natl Acad. Sci. USA 89: 8794-7; Kim et al. (1994) Genomics 22: 336-9; Woo et al. (1994) Nucleic Acids Res. 22(23): 4922-31; Wang et al. (1995) Plant 3: 525-33; Cai (1995) Genomics 29(2): 413-25; Schmitt et al. (1996) Genomics 33: 9-20; Kim et al. (1996) Genomics 34(2): 213-8; Kim et al. (1996) Proc. Natl. Acad. Sci. USA 13: 6297-301; Pusch et al., (1996) Gene 183(1-2): 29-33; and Wang et al. (1996) Genome Res. 6(7): 612-9. Improved methods of in vitro amplification to amplify large nucleic acids linked to the polymorphic nucleic acids herein are summarized in Cheng et al. (1994) Nature 369: 684-685 and the references therein.

In addition, any of the cloning or amplification strategies described herein are useful for creating contigs of overlapping clones, thereby providing overlapping nucleic acids which show the physical relationship at the molecular level for genetically linked nucleic acids. A common example of this strategy is found in whole organism sequencing projects, in which overlapping clones are sequenced to provide the entire sequence of a chromosome. In this procedure, a library of the organism's cDNA or genomic DNA is made according to standard procedures described, e.g., in the references above. Individual clones are isolated and sequenced, and overlapping sequence information is ordered to provide the sequence of the organism. See also, Tomb et al. (1997) Nature 388: 539-547 describing the whole genome random sequencing and assembly of the complete genomic sequence of Helicobacter pylori; Fleischmann et al. (1995) Science 269: 496-512 describing whole genome random sequencing and assembly of the complete Haemophilus influenzae genome; Fraser et al. (1995) Science 270: 397-403 describing whole genome random sequencing and assembly of the complete Mycoplasma genitalium genome; and Bult et al. (1996) Science 273: 1058-1073 describing whole genome random sequencing and assembly of the complete Methanococcus jannaschii genome. Hagiwara and Curtis, Nucleic Acids Res. 24: 2460-2461 (1996) developed a “long distance sequencer” PCR protocol for generating overlapping nucleic acids from very large clones to facilitate sequencing, and methods of amplifying and tagging the overlapping nucleic acids into suitable sequencing templates. The methods can be used in conjunction with shotgun sequencing techniques to improve the efficiency of shotgun methods typically used in whole organism sequencing projects. As applied to the present invention, the techniques are useful for identifying and sequencing genomic nucleic acids genetically linked to the QTLs as well as “candidate” genes responsible for QTL expression as identified by the methods herein. As noted above, the allelic sequences that comprise a QTL can be cloned and inserted into a transgenic plant. Methods of creating transgenic plants are well known in the art and are described in brief below.

Transgenic Plants

Nucleic acids derived from those linked to a genetic marker and/or QTL identified by the statistical methods herein can be introduced into plant cells, either in culture or in organs of a plant, e.g., leaves, stems, fruit, seed, etc. The expression of natural or synthetic nucleic acids can be achieved by operably linking a nucleic acid of interest to a promoter, incorporating the construct into an expression vector, and introducing the vector into a suitable host cell.

Typical vectors (e.g., plasmids) contain transcription and translation terminators, transcription and translation initiation sequences, and/or promoters useful for regulation of the expression of the particular nucleic acid. The vectors optionally comprise generic expression cassettes containing promoter, gene, and terminator sequences, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, e.g., Berger; Sambrook; and Ausubel.

Cloning of QTL Allelic Sequences into Bacterial Hosts

Bacterial cells can be used to increase the number of plasmids containing the DNA constructs of this invention. The plasmids can be introduced into bacterial host cells by any of a number of methods known in the art (e.g., electroporation or calcium chloride). The bacteria are grown, and the plasmids within the bacteria are isolated by a variety of methods known in the art (see, for instance, Sambrook). In addition, a plethora of kits are commercially available for the purification of plasmids from bacteria (for example, StrataClean™ from Stratagene or QIAprep™ from Qiagen). The isolated and purified plasmids can then be further manipulated to produce other plasmids, used to transfect plant cells, or incorporated into Agrobacterium tumefaciens to infect plants.

Alternatively, a cloned plant nucleic acid can be expressed in bacteria such as E. coli and the resulting protein can be isolated and purified.

Transfecting Plant Cells

Preparation of Recombinant Vectors

To use isolated sequences in the above techniques, recombinant DNA vectors suitable for transformation of plant cells are prepared. Techniques for transforming a wide variety of higher plant species are well known and described in the technical and scientific literature. See, for example, Weising et al. (1988) Ann. Rev. Genet. 22: 421-477. A DNA sequence coding for a desired polypeptide (for example, a cDNA sequence encoding a full length protein) will preferably be combined with transcriptional and translational initiation regulatory sequences which will direct the transcription of the sequence from the gene.

Promoters can be identified by analyzing the 5′ sequences upstream of the coding sequence of an allele associated with a QTL. Sequences characteristic of promoter sequences can be used to identify the promoter. Sequences controlling eukaryotic gene expression have been extensively studied. For instance, promoter sequence elements include the TATA box consensus sequence (TATAAT), which is usually 20 to 30 base pairs upstream of the transcription start site. In most instances the TATA box is required for accurate transcription initiation. In plants, further upstream from the TATA box, at positions −80 to −100, there is typically a promoter element with a series of adenines surrounding the trinucleotide G (or T) N G. See, e.g., J. Messing et al. (1983) in Genetic Engineering in Plants, pp. 221-227 (Kosage, Meredith and Hollaender, eds.). A number of methods are known to those of skill in the art for identifying and characterizing promoter regions in plant genomic DNA (see, e.g., Jordano et al. (1989) Plant Cell 1: 855-866; Bustos et al. (1989) Plant Cell 1: 839-854; Green et al. (1988) EMBO J. 7: 4035-4044; Meier et al. (1991) Plant Cell 3: 309-316; and Zhang et al. (1996) Plant Physiology 110: 1069-1079).

In construction of recombinant expression cassettes of the invention, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35 S transcription initiation region, the ubiquitin promoter, the 1′- or 2′-promoter derived from T-DNA of Agrobacterium tumefaciens, and other transcription initiation regions from various plant genes known to those of skill.

Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers. For example, the tissue specific E8 promoter from tomato is useful for directing gene expression so that a desired gene product is located in fruits. Other suitable promoters include those from genes encoding embryonic storage proteins. Examples of environmental conditions that may affect transcription by inducible promoters include anaerobic conditions, elevated temperature, or the presence of light.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from QTL alleles of the invention will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon or glufosinate.

Introduction of the Nucleic Acids into Plant Cells

The DNA constructs of the invention can be introduced into plant cells, either in culture or in the organs of a plant, by a variety of conventional techniques. For example, the DNA construct can be introduced directly into the plant cell using techniques such as electroporation and microinjection of plant cell protoplasts, or the DNA constructs can be introduced directly to plant cells using ballistic methods, such as DNA particle bombardment. Alternatively, the DNA constructs are combined with suitable T-DNA flanking regions and introduced into a conventional Agrobacterium tumefaciens host vector. The virulence functions of the Agrobacterium tumefaciens host directs the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski et al. (1984) EMBO J. 3: 2717. Electroporation techniques are described in Fromm et al. (1985) Proc. Nat'l Acad. Sci. USA 82: 5824. Ballistic transformation techniques are described in Klein et al. (1987) Nature 327: 70-73. Agrobacterium tumefaciens-mediated transformation techniques, including disarming and use of binary vectors, are also well described in the scientific literature. See, for example Horsch et al. (1984) Science 233: 496-498 and Fraley et al. (1983) Proc. Nat'l Acad. Sci. USA 80: 4803.

Generation of Transgenic Plants

Transformed plant cells (e.g., those derived by any of the above transformation techniques) can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al. (1983) “Protoplasts Isolation and Culture” in the Handbook of Plant Cell Culture, pp. 124-176, Macmillian Publishing Company, N.Y.; and Binding (1985) Regeneration of Plants, Plant Protoplasts, pp. 21-73, CRC Press, Boca Raton. Regeneration can also be obtained from plant callus, explants, somatic embryos (e.g., Dandekar et al. (1989) J. Tissue Cult. Meth. 12: 145 and McGranahan et al. (1990) Plant Cell Rep. 8: 512), organs, or parts thereof. Such regeneration techniques are described generally in Klee et al. (1987) Ann. Rev. of Plant Phys. 38: 467-486.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

### EXAMPLES

The following sets forth a series of experiments that demonstrate determination and use of an association between cob color and a genetic marker haplotype in maize. It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

Cob color (e.g., red or white) in maize is determined in part by the pericarp color 1 (p1) gene. See, e.g., Neuffer, Coe, and Wessler (1997) Mutants of Maize, Cold Spring Harbor Laboratory Press, p 107 for a description of p1-wr, p 363 for a description of the gene and its mode of action, and p 35 for its map location. The following example describes determination of an association between cob color and a genetic marker sequence that is linked to p1.

Linkage Map

To generate genetic marker information, a large number of loci selected from an EST database were sequenced across a set of inbreds chosen from a multigeneration pedigree (Pioneer's established maize breeding population). These markers were used to generate a multipoint linkage map basically as follows.

The set of genetic markers included 5741 haplotypes (haplotype blocks) generated by sequencing approximately 450 base pairs from each of 5741 EST sequences from each of the inbreds. For example, marker MZA6914 haplotype was genotyped by sequencing a nested PCR product amplified using the following primers: outer primers taggtgctttgcggaccttg (SEQ ID NO:1) and tctgaacagcaaatcgttgttg (SEQ ID NO:2), and inner primers aggaaacagctatgaccat (SEQ ID NO:3) and gttttcccagtcacgacg (SEQ ID NO:4). The set of genetic markers also included 505 SSR markers that had been genotyped in B73/Mol7 and mapped on the public IBM2 map.

The set of inbreds chosen from the established breeding population included 320 triplets, each containing two inbred lines and a third inbred line derived from a cross between those two lines, corresponding to about 600 inbreds total. Using pedigree information and triplets containing inbred parents having different marker alleles, a multipoint linkage map containing the 6246 markers (5741 haplotypes and 505 SSRs) was developed by assigning the markers to chromosomes and ordering the markers on the chromosomes. (It will be evident that not every triplet is informative for every marker, e.g., if the parents have the same marker allele). The linkage map used the public IBM2 map (http://www.maizegdb.org) as the backbone. Overgo probes were designed for most of the 5741 sequenced loci and hybridized to a physical map, helping link the physical and genetic maps and permitting markers that were too close to genetically map to be ordered.

Likelihood Ratio TDT Test

Phenotypic data (red or white cob color) for the inbred lines used to generate the linkage map had been collected as part of Pioneer's ongoing breeding program. Association analysis was performed using the third inbred from triplets in which the two parental inbred lines had different phenotypes for cob color (i.e., one red parent and one white parent); the third inbreds from these triplets, chosen from the established breeding population, comprise the first plant population. The set of genetic markers included 511 markers on chromosome 1 (488 haplotypes and 23 SSRs) whose genotypes had been determined by sequencing as noted above. (The analysis was limited to the first chromosome since the p1 locus is on chromosome 1.) Again, it will be evident that not every triplet is informative for every marker; only triplets in which the inbred parents have different marker haplotypes are informative. The genetic marker and phenotypic information, along with pedigree relationships between the inbreds in the first plant population, were used in a TDT analysis (see, e.g., Gutin et al. (2001) “Allelic association in large pedigrees” Genet Epidemiol. 21 Suppl 1: S571-575 and Spielman et al. (1993) “Transmission test for linkage disequilibrium: The insulin gene region and insulin-dependent diabetes mellitus (IDDM)” American Journal of Human Genetics 52: 506-516).

A TDT-based association test using haplotype data in which each haplotype can have more than two alleles can be computed from a TDT test for multiple alleles (originally proposed by Spielman and Ewens (1996) “The TDT and other family-based tests for linkage disequilibrium and association” American Journal of Human Genetics 59: 983-989) converted into a likelihood ratio test, which will be referred to as a Likelihood Ratio TDT Test (LR-TDT). We first briefly describe the test for bi-allele marker data and then extend the method to the analysis of multiple allele data.

For bi-allele data, we define the conditional probabilities of transmitting allele M₁ and not transmitting allele M₂ given parental genotype M₁M₂ to be t₁₂=P(M₁,M₂|g=M₁M₂) and of transmitting allele M₂ but not M₁ be t₂₁=P(M₂,M₁|g=M₁M₂). The maximum likelihood estimates of t₁₂ and t₂₁ are n₁₂/(n₁₂+n₂₁) and n₂₁/(n₁₂+n₂₁), respectively. There are n individuals with informative parents for the marker of interest; n₁₂ of these inherited the first marker allele and the second trait phenotype, and n₂₁ of these inherited the second marker allele and the first trait phenotype. The log-likelihood function of transmitting a marker allele from heterozygous parents to affected offspring is then

$$ln {L}_{1} = {n}_{12} ln ( {t}_{12} ) + {n}_{21} ln ( {t}_{21} ) = {n}_{12} ln \frac{{n}_{12}}{{n}_{12} + {n}_{21}} + {n}_{21} ln \frac{{n}_{21}}{{n}_{12} + {n}_{21}} .$$

The corresponding log-likelihood function at the null hypothesis is

$$ln {L}_{0} = ( {n}_{12} + {n}_{21} ) ln \frac{1}{2} .$$

The likelihood ratio test statistic is
LRT=2(ln L₁−ln L₀);
it has a chi-square distribution with df=1 (df represents degrees of freedom).

To extend the above formula to multiple allele marker data, we assume k alleles for each marker locus (each marker haplotype in this example). We designate one allele, Mᵥ, as the M₁ allele. All other alleles are treated together as allele M₂, and their allele counts are pooled so the multiple allele data is converted into k bi-allele data sets. The log likelihood ratio test statistic for k alleles (LRTₖ) is thus the sum of k independent log likelihood ratio tests (LRTᵥ):

$${LRT}_{k} = \frac{k - 1}{k} {\sum }_{v = 1}^{k} {LRT}_{k} = \frac{k - 1}{k} {\sum }_{v = 1}^{k} 2 ( ln {L}_{v1} - ln {L}_{v0} ) .$$

The above multiple allele log likelihood ratio test statistic has an asymptotic chi-square distribution with degree of freedom df=k−1.

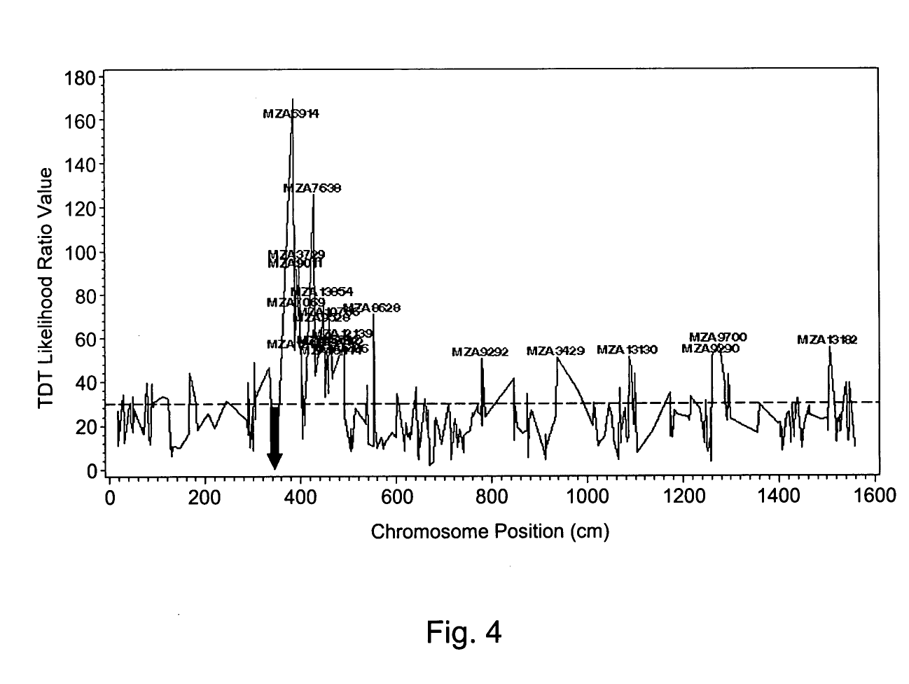

FIG. 4 plots the TDT likelihood ratio statistic for cob color for the 511 markers ordered by chromosome position. The horizontal dashed line on the likelihood profile (FIG. 4) is the threshold or significant LRTₖ value after Bonferroni adjustment for multiple loci testing α_{b}=α/m, where m is the number of markers on the chromosome and α=0.01. The arrow indicates the position of the p1 locus. Map positions are given with respect to the multipoint linkage map described above.

Table 1 presents additional details about the LR-TDT test. For each of several genetic marker haplotypes (indicated by an MZA number), the table indicates the sample size (number of third inbreds in the first plant population, corresponding to the number of triplets informative for the particular marker), degrees of freedom (df, equal to the number of marker haplotypes minus one), chi-square value for the TDT test, the probability associated with that chi-square value, linkage group (corresponding to the public maize genetic map), and map position in centimorgans (cm, with respect to the multipoint linkage map described above). Note that genetic marker haplotypes with a frequency of less than 5% were not included in the analysis. For MZA6914, for example, three haplotypes each had a frequency less than 5% and were not considered while three haplotypes each had a frequency greater than 5% and were considered.

TABLE 1
LR-TDT results for cob color.

| trait | marker | sample size | df | Z_Chi_sq | Pval_Z_CHIsq | linkage group | position |
|---|---|---|---|---|---|---|---|
| RED | MZA6914 | 100 | 3 | 49.08 | 0 | 1.03 | 385.69 |
| RED | MZA1241 | 230 | 4 | 14.74 | 4.38E−07 | 1.03 | 389.00 |
| RED | MZA9011 | 246 | 7 | 22.68 | 9.51E−07 | 1.03 | 391.98 |
| RED | MZA7069 | 250 | 7 | 18.29 | 3.13E−09 | 1.03 | 394.18 |
| RED | MZA3729 | 282 | 7 | 23.72 | 9.14E−10 | 1.03 | 396.25 |

As indicated in FIG. 4 and Table 1, a highly significant association is observed between marker MZA6914 and cob color. MZA6914 is not the p1 gene but is a sequence tightly linked to p1, based on information from the physical map.

Applications

From the association between MZA6914 and cob color determined in the first population of inbreds as described above, cob color can be predicted in other plants based on their MZA6914 genotype, and this information can be applied to selection and breeding for desired phenotypes. For example, plants having the desired MZA6914 genotype (e.g., a MZA6914 haplotype associated with white cobs) can be identified before pollination and used as parents in white corn product development programs, e.g., where their offspring (comprising the target plant population) are predicted to have white cobs. White cob color is desired, for example, in hybrids having white kernels, since red glumes are difficult to remove and can add undesirable color to corn chips, tortillas, etc. produced from the kernels. Selection for plants before pollination can result in significant labor savings in the development process. Prediction of an offspring's cob color phenotype prior to pollination of the plants can thus increase the efficiency of developing inbred lines and/or hybrids having white cobs and white kernels.

The association can, if desired, be verified in segregating crosses prior to use in selecting parents and predicting offspring phenotypes in a breeding program.

The example of association analysis and phenotypic trait prediction described above uses cob color, but this type of analysis and prediction is equally applicable to any qualitative trait or any simple trait conditioned by a single gene. For example, single genes condition resistance to a number of plant diseases, and the strategy outlined in this example can be used to predict, breed and/or select for offspring resistant to such diseases. A number of other examples of simple traits are provided in Mutants of Maize (supra).

Also as noted herein, related strategies can be applied to determining associations and predicting phenotypes for traits that have a continuous phenotypic distribution and that may be controlled by multiple loci, by using statistical analysis designed to identify genetic regions associated with continuous traits.

While the foregoing invention has been described in some detail for purposes of clarity and understanding, it will be clear to one skilled in the art from a reading of this disclosure that various changes in form and detail can be made without departing from the true scope of the invention. For example, all the techniques and compositions described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of predicting a value of a phenotypic trait in a target plant population, the method comprising:
(a) providing an association between at least one genetic marker and the phenotypic trait; wherein the association is evaluated in a first plant population, the first plant population being an established breeding population or a portion thereof; wherein the association is evaluated in the first plant population according to a statistical model that incorporates a genotype of the first plant population for a set of genetic markers and a value of the phenotypic trait in the first plant population; and,
(b) providing the value of the phenotypic trait in at least one member of the target plant population, wherein the providing comprises predicting the value from the association of (a) and from a genotype of the at least one member for the at least one genetic marker associated with the phenotypic trait.

2. The method of claim 1, wherein the first plant population comprises a plurality of inbreds, single cross F1 hybrids, or a combination thereof.

3. The method of claim 2, wherein the first plant population consists of inbreds, single cross F1 hybrids, or a combination thereof.

4. The method of claim 2, wherein the ancestry of each inbred and/or single cross F1 hybrid is known, and wherein each inbred and/or single cross F1 hybrid is a descendent of at least one of three or more founders.

5. The method of claim 1, wherein the established breeding population comprises at least three founders and descendents of the founders, wherein the ancestry of the descendents is known.

6. The method of claim 5, wherein the established breeding population comprises between about 100 and about 200 founders and descendents of the founders, wherein the ancestry of the descendents is known.

7. The method of claim 1, wherein the members of the first plant population span at least three breeding cycles.

8. The method of claim 7, wherein the members of the first plant population span at least four breeding cycles.

9. The method of claim 7, wherein the members of the first plant population span at least seven or at least nine breeding cycles.

10. The method of claim 1, wherein the phenotypic trait is a quantitative phenotypic trait.

11. The method of claim 1, wherein the phenotypic trait is a qualitative phenotypic trait.

12. The method of claim 1, further comprising selecting at least one of the members of the target plant population having a desired predicted value of the phenotypic trait.

13. The method of claim 12, further comprising breeding at least one selected member of the target plant population with at least one other plant.

14. The method of claim 1, wherein the first plant population comprises between about 50 and about 5000 members.

15. The method of claim 1, wherein the first plant population comprises a plurality of inbreds.

16. The method of claim 1, wherein the first plant population comprises a plurality of single cross F1 hybrids.

17. The method of claim 1, wherein the first plant population comprises a plurality of a combination of inbreds and single cross F1 hybrids.

18. The method of claim 1, wherein the value of the phenotypic trait in the first plant population is obtained by evaluating the phenotypic trait among the members of the first plant population in at least one topcross combination with at least one tester parent.

19. The method of claim 1, wherein the phenotypic trait is selected from the group consisting of: yield, grain moisture content, grain oil content, root lodging resistance, stalk lodging resistance, plant height, ear height, disease resistance, insect resistance, drought resistance, grain protein content, test weight, and cob color.

20. The method of claim 1, wherein the set of genetic markers comprises one or more of: a single nucleotide polymorphism (SNP), a multinucleotide polymorphism, an insertion of at least one nucleotide, a deletion of at least one nucleotide, a simple sequence repeat (SSR), a restriction fragment length polymorphism (RFLP), a random amplified polymorphic DNA (RAPD) marker, or an arbitrary fragment length polymorphism (AFLP).

21. The method of claim 1, wherein the set of genetic markers comprises between one and ten markers.

22. The method of claim 1, wherein the set of genetic markers comprises between 500 and 50,000 markers.

23. The method of claim 1, wherein the genotype of the first plant population for the set of genetic markers is obtained by experimentally determining the genotype of each inbred and predicting the genotype of each single cross F1 hybrid present in the first plant population.

24. The method of claim 23, wherein experimentally determining the genotype of each inbred comprises sequencing a set of DNA segments from each inbred.

25. The method of claim 24, wherein the set of DNA segments comprises the 5′-untranslated regions and/or the 3′-untranslated regions of two or more genes.

26. The method of claim 1, wherein providing the association between at least one genetic marker and the phenotypic trait comprises providing an association between a haplotype comprising two or more genetic markers and the phenotypic trait.

27. The method of claim 1, wherein the statistical model incorporates family relationships among the members of the first plant population.

28. The method of claim 1, wherein evaluating the association according to the statistical model comprises performing Bayesian analysis using a linear model, a mixed linear model, or a nonlinear model.

29. The method of claim 28, wherein the Bayesian analysis is implemented via a reversible jump Markov chain Monte Carlo algorithm, a delta method, or a profile likelihood algorithm.

30. The method of claim 1, wherein evaluating the association according to the statistical model comprises performing Bayesian analysis using a linear model, the Bayesian analysis being implemented via a reversible jump Markov chain Monte Carlo algorithm.

31. The method of claim 1, wherein evaluating the association according to the statistical model comprises performing a transmission disequilibrium test.

32. The method of claim 1, wherein evaluating the association comprises and/or permits determining identity by descent information for founder alleles of the at least one genetic marker in one or more pedigrees of related inbreds and/or single cross F1 hybrids, and permits tracking of the at least one genetic marker throughout such pedigrees.

33. The method of claim 1, wherein the genotype of the at least one member of the target plant population for the at least one genetic marker is determined experimentally.

34. The method of claim 33, wherein the genotype is determined experimentally by high throughput screening.

35. The method of claim 1, wherein the genotype of the at least one member of the target plant population for the at least one genetic marker is predicted.

36. The method of claim 1, wherein the target plant population comprises inbred plants.

37. The method of claim 1, wherein the target plant population comprises hybrid plants.

38. The method of claim 37, wherein the hybrid plants comprise F1 progeny produced from single crosses between inbred lines.

39. The method of claim 38, wherein the F1 progeny are produced from single crosses between inbreds comprising the first plant population, the hybrid plants not comprising the first plant population.

40. The method of claim 1, wherein the target plant population comprises an advanced generation produced from breeding crosses comprising at least one of the members of the first plant population.

41. The method of claim 1, wherein predicting the value of the phenotypic trait in the at least one member of the target plant population comprises predicting the value using a best linear unbiased prediction method.

42. The method of claim 1, wherein predicting the value of the phenotypic trait in the at least one member of the target plant population comprises predicting the value using a multiple regression method, a selection index technique, a ridge regression method, a linear optimization method, or a non-linear optimization method.

43. The method of claim 1, wherein the first and target plant populations consist of diploid plants.

44. The method of claim 1, wherein the first and target plant populations are selected from the group consisting of: maize, soybean, sorghum, wheat, sunflower, rice, canola, cotton, and millet.

45. The method of claim 44, wherein the first and target plant populations comprise maize.

46. The method of claim 45, wherein the first and target plant populations comprise Zea mays.

47. The method of claim 1, further comprising cloning a gene that is linked to the at least one genetic marker associated with the phenotypic trait, wherein expression of the gene affects the phenotypic trait.

48. The method of claim 47, further comprising constructing a transgenic plant by expressing the cloned gene in a host plant.

49. A plant selected by the method of claim 12.

50. A plant produced by the breeding method of claim 13.

51. A transgenic plant created by the method of claim 48.

52. A method of selecting a plant, the method comprising:
(a) providing an association between at least one genetic marker and the phenotypic trait; wherein the association is evaluated in a first plant population, the first plant population being an established breeding population or a portion thereof; wherein the association is evaluated in the first plant population according to a statistical model that incorporates a genotype of the first plant population for a set of genetic markers and a value of the phenotypic trait in the first plant population; and,
(b) providing one or more plants from one or more non-adapted lines, wherein the providing comprises selecting one or more plants for a selected genotype comprising the at least one genetic marker associated with the phenotypic trait.

53. The method of claim 52, wherein the first plant population comprises a plurality of inbreds, single cross F1 hybrids, or a combination thereof.

54. The method of claim 53, wherein the first plant population consists of inbreds, single cross F1 hybrids, or a combination thereof.

55. The method of claim 53, wherein the ancestry of each inbred and/or single cross F1 hybrid is known, and wherein each inbred and/or single cross F1 hybrid is a descendent of at least one of three or more founders.

56. The method of claim 52, wherein the established breeding population comprises at least three founders and descendents of the founders, wherein the ancestry of the descendents is known.

57. The method of claim 56, wherein the established breeding population comprises between about 100 and about 200 founders and descendents of the founders, wherein the ancestry of the descendents is known.

58. The method of claim 52, wherein the members of the first plant population span at least three breeding cycles.

59. The method of claim 58, wherein the members of the first plant population span at least four breeding cycles.

60. The method of claim 58, wherein the members of the first plant population span at least seven or at least nine breeding cycles.

61. The method of claim 52, wherein the phenotypic trait is a quantitative phenotypic trait.

62. The method of claim 52, wherein the phenotypic trait is a qualitative phenotypic trait.

63. The method of claim 52, further comprising evaluating the phenotypic trait in the one or more plants having the selected genotype.

64. The method of claim 63, further comprising selecting at least one plant having the selected genotype and a desirable value of the phenotypic trait.

65. The method of claim 64, further comprising breeding the at least one selected plant having the selected genotype and the desirable value of the phenotypic trait with at least one other plant.

66. The method of claim 52, wherein the value of the phenotypic trait in the first plant population is obtained by evaluating the phenotypic trait among the members of the first plant population in at least one topcross combination with at least one tester parent.

67. The method of claim 52, wherein the phenotypic trait is selected from the group consisting of: yield, grain moisture content, grain oil content, root lodging resistance, stalk lodging resistance, plant height, ear height, disease resistance, insect resistance, drought resistance, grain protein content, test weight, and cob color.

68. The method of claim 52, wherein the set of genetic markers comprises one or more of: a single nucleotide polymorphism (SNP), a multinucleotide polymorphism, an insertion of at least one nucleotide, a deletion of at least one nucleotide, a simple sequence repeat (SSR), a restriction fragment length polymorphism (RFLP), a random amplified polymorphic DNA (RAPD) marker, or an arbitrary fragment length polymorphism (AFLP).

69. The method of claim 52, wherein the genotype of the first plant population for the set of genetic markers is obtained by experimentally determining the genotype of each inbred and predicting the genotype of each single cross F1 hybrid present in the first plant population.

70. The method of claim 69, wherein experimentally determining the genotype of each inbred comprises sequencing a set of DNA segments from each inbred.

71. The method of claim 70, wherein the set of DNA segments comprises the 5′-untranslated regions and/or the 3′-untranslated regions of two or more genes.

72. The method of claim 52, wherein providing the association between at least one genetic marker and the phenotypic trait comprises providing an association between a haplotype comprising two or more genetic markers and the phenotypic trait.

73. The method of claim 52, wherein the statistical model incorporates family relationships among the members of the first plant population.

74. The method of claim 52, wherein evaluating the association according to the statistical model comprises performing Bayesian analysis using a linear model, a mixed linear model, or nonlinear model.

75. The method of claim 74, wherein the Bayesian analysis is implemented via a reversible jump Markov chain Monte Carlo algorithm, a delta method, or a profile likelihood algorithm.

76. The method of claim 52, wherein evaluating the association according to a statistical model comprises performing Bayesian analysis using a linear model, the Bayesian analysis being implemented via a reversible jump Markov chain Monte Carlo algorithm.

77. The method of claim 52, wherein evaluating the association according to the statistical model comprises performing a transmission disequilibrium test.

78. The method of claim 52, wherein the first plant population and the one or more non-adapted lines consist of diploid plants.

79. The method of claim 52, wherein the first plant population and the one or more non-adapted lines are selected from the group consisting of: maize, soybean, sorghum, wheat, sunflower, rice, canola, cotton, and millet.

80. The method of claim 79, wherein the first plant population and the one or more non-adapted lines comprise maize.

81. The method of claim 80, wherein the first plant population and the one or more non-adapted lines comprise Zea mays.

82. The method of claim 64, further comprising cloning a gene that is linked to the at least one genetic marker associated with the phenotypic trait from the at least one selected plant having the selected genotype and the desirable value of the phenotypic trait, wherein expression of the gene affects the phenotypic trait.

83. The method of claim 82, further comprising constructing a transgenic plant by expressing the cloned gene in a host plant.

84. A plant provided by the method of claim 52.

85. A plant selected by the method of claim 64.

86. A plant produced by the breeding method of claim 65.

87. A transgenic plant created by the method of claim 83.


SEQUENCE LISTING (4 sequences)

SEQ ID NO: 1 - DNA, 20 bp, Artificial
  oligonucleotide primer
taggtgctttgcggaccttg

SEQ ID NO: 2 - DNA, 22 bp, Artificial
  oligonucleotide primer
tctgaacagcaaatcgttgttg

SEQ ID NO: 3 - DNA, 19 bp, Artificial
  oligonucleotide primer
aggaaacagctatgaccat

SEQ ID NO: 4 - DNA, 18 bp, Artificial
  oligonucleotide primer
gttttcccagtcacgacg